(19) United States
(12) Patent Application Publication
(10) Pub. No.: US 2022/0256900 A1
(43) Pub. Date: Aug. 18, 2022

(54) COMPOSITIONS WITH SUGAR LIKE CHARACTERISTICS

(71) Applicants: CORN PRODUCTS DEVELOPMENT, INC., Westchester, IL (US); CHROMOCELL CORPORATION, North Brunswick, NJ (US)

(72) Inventors: Kasi SUNDARESAN, Bridgewater, NJ (US); Purvi SHAH, Bridgewater, NJ (US)

(21) Appl. No.: 17/618,570

(22) PCT Filed: Jun. 9, 2020
(86) PCT No.: PCT/US2020/036779

§ 371 Date: Dec. 13, 2021

Related U.S. Application Data
(60) Provisional application No. 62/860,701, filed on Jun. 12, 2019.

(51) Int. Cl. A23L 27/30; A23L 27/00

(52) U.S. Cl. CPC A23L 27/30; C07D 209/14; A23L 27/88; A23L 27/84

(57) ABSTRACT

Disclosed herein are sweetener compositions comprising at least one compound of Formula (I) and at least one additional sweetener to provide sugar-like characteristics. Beneficially, the sweetener compositions provide characteristics like that of sucrose that include maximal response, flavor profile, temporal profile, adaptation behavior and mouthfeel. The sweetener compositions can be included in various food, beverage, and other consumable products to provide a clean sugar-like taste and may be used as sweeteners or sweetness enhancers in reduced sugar food, beverage, and other consumable products.

### CROSS-REFERENCE TO RELATED APPLICATION

This application claims the benefit of U.S. Provisional Patent Application No. 62/860,701, filed Jun. 12, 2019, which is hereby incorporated herein by reference in its entirety.

Disclosed herein are sweetener compositions comprising at least one compound of Formula (I) and at least one additional sweetener to provide ‘sugar like’ characteristics. Beneficially, the sweetener compositions provide characteristics like that of sucrose that include maximal response, flavor profile, temporal profile, adaptation behavior and mouthfeel. The sweetener compositions can be included in various food, beverage, and other consumable products to provide a clean sugar-like taste and may be used as sweeteners or sweetness enhancers in reduced sugar food. beverage, and other consumable products.

Sugars and sweeteners are widely used in food, beverage, and other consumable products and play a critical role in consumer's selection and enjoyment of such products. Sugars are desirable for their sweet taste and/or the flavor they elicit. Sucrose imparts a taste preferred by consumers that is the ‘gold standard’ in terms of the desired onset (appearance time) and decay of sweetness (extinction time). Although sucrose provides superior sweetness characteristics, it is caloric. Non-caloric or low caloric sweeteners, both nutritive and non-nutritive, have been introduced to satisfy consumer demand for sweet tasting products that provide with non-caloric or low-caloric sweetness.

A key challenge in replacing sucrose with non-nutritive high potency sweeteners is to achieve a sweetness profile like sucrose. In addition, sweeteners can be challenging to formulate into products such as foods and beverages. Sweeteners cannot simply be substituted into products that contain sugars. Various formulation and processing challenges can exist, including finding the desirable sugar taste when using combined sugars and/or sweeteners, along with removing any undesirable taste properties that they can impart. On a taste basis, non-caloric or low caloric sweeteners exhibit a temporal profile, maximal response, flavor profile, mouthfeel, and/or adaptation behavior that differ from sugar. Specifically, non-caloric or low caloric sweeteners exhibit delayed sweetness onset, lingering sweet aftertaste, bitter taste, metallic taste, astringent taste, cooling taste and/or licorice-like taste.

Accordingly, there is an ongoing demand for sweetener compositions that provide ‘sugar like’ characteristics, including the maximal response, flavor profile (i.e., lack of detectable off tastes (e.g., metallic, licorice and bitter notes)), cooling and adaptation behaviors of sucrose. There is also a mouthfeel aspect that's characteristic of “sugar like” taste. There is a clear consumer desire for natural non-caloric and/or low caloric sweeteners that have the same taste quality as sucrose.

There is also a further need to develop natural reduced or non-caloric sweeteners (also referred to as low calorie and/or zero calorie sweeteners) that provide temporal and flavor profile similar to that of sucrose. This need is to replace detectable off tastes (e.g., metallic, licorice and bitter notes), cooling, and adaptation behaviors of high potency sweeteners while still providing a natural non-caloric sweetener that has the same taste quality as sucrose.

In addition, there remains a further need to develop sweetened compositions, such as foods, beverage, and/or other consumable products containing reduced or non-caloric sweetener compositions having more sugar-like characteristics than currently available reduced or non-caloric sweetener compositions.

### SUMMARY

Disclosed herein are sweetener compositions and food, beverage, and/or other consumable products containing said sweetener compositions. The sweetener compositions described herein have ‘sugar like’ properties and can beneficially be included in various food, beverage, and/or other consumable products to provide a clean sugar-like taste while providing reduced sugar food, beverage, and other consumable products.

In one embodiment, the sweetener composition comprises: (a) at least one compound of Formula (I) in an effective amount to provide a sucrose sweetness equivalence of at least 1% to the sweetener composition:

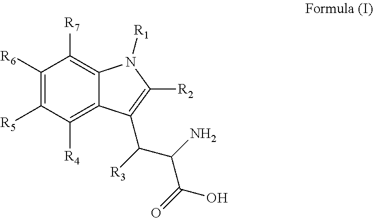

or a comestibly or biologically acceptable salt or stereoisomeric form thereof, wherein: R1 is hydrogen or C1-C6 alkyl; R2-R5 and R7 are each independently hydrogen, halogen, C1-C6 alkyl, C1-C6 alkoxy, hydroxyl or trifluoromethyl; and R6 is I, hydrogen, C4-C6 alkyl, C4-C6 alkoxy or hydroxyl; and (b) at least one additional sweetener comprising a fully caloric, low calorie, and/or zero calorie sweetener, wherein the sweetener composition provides more sugar-like characteristics than a sweetener composition without the compound of Formula (I).

In a further embodiment, the consumable food, beverage, or other product comprises: a concentration from about 5 ppm to about 4000 ppm of one or more sweetener composition described herein; at least one additive, bulking agent and/or functional ingredient; and a food, beverage, or other consumable composition, wherein the consumable food, beverage, or other product has an equivalent sweetness to sucrose solutions having concentrations between 10,000 and 400,000 ppm (1-40 sucrose sweetness equivalent). In some embodiments, the sweetener compound has the following structure:

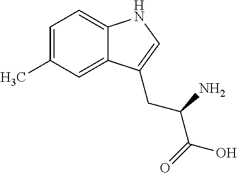 [SMILES: Cc1ccc2[nH]cc(C[C@@H](N)C(=O)O)c2c1]

In a further embodiment, a method for enhancing the sugar-like characteristics of a consumable food, beverage, or other product comprises providing a consumable food, beverage, or other product composition that is sweetenable; and adding to the product one or more sweetener composition described herein.

Through various embodiments described herein, one may produce and use various sweetener compositions that impart desirable levels of sweetness along with ‘sugar like’ properties, including maximal response, flavor profile, adaptation behavior and mouthfeel. These sweetener compositions may be used as sweeteners in many applications, including but not limited to food products, pharmaceutical compositions, edible gel mixes and compositions, dental compositions, confections, condiments, chewing gum, cereal compositions, baked goods, dairy products, tabletop sweeteners, beverage products, fruits preparations and concentrates, dressings, frozen food, canned food, desserts, bars, snacks, sauces, and spreads.

### BRIEF DESCRIPTION OF THE FIGURES

FIG. 1 shows the concentration relationship between sucrose and the Test Compound Sweetener using a dose response model as described in Example 1.

FIG. 2 shows the rating scale for the sweetness intensity on the gLMS scale to evaluate the different intensities of sweetness evaluated in Example 1.

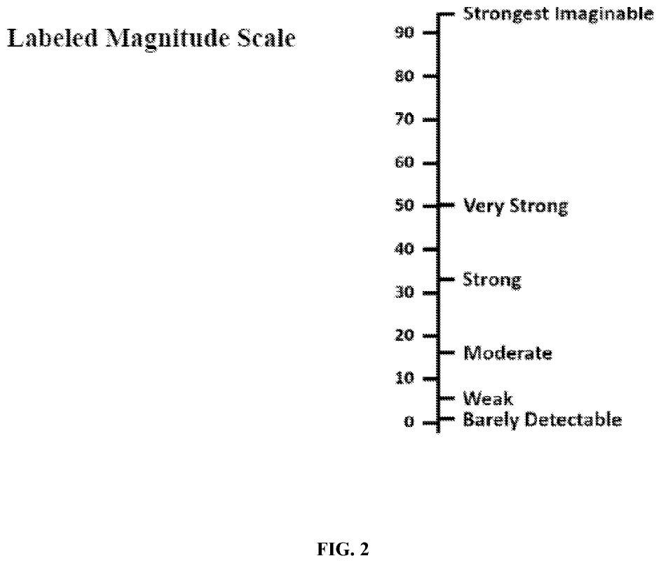

FIG. 3 shows the sweetness intensity scaling for blends of the Test Compound Sweetener and Reb M as evaluated in Example 2.

Reference will now be made in detail to various embodiments of the present invention, examples of which are illustrated in the accompanying figure. In the following description, numerous specific details are set forth in order to provide a thorough understanding of the present invention. However, unless otherwise indicated or implicit from context, the details are intended to be examples and should not be deemed to limit the scope of the invention in any way. Additionally, features described in connection with the various or specific embodiments are not to be construed as not appropriate for use in connection with other embodiments disclosed herein unless such exclusivity is explicitly stated or implicit from context.

All terminology used herein is for the purpose of describing particular embodiments only and is not intended to be limiting in any manner or scope. For example, as used in this specification and the appended claims, the singular forms “a,” “an” and “the” can include plurals unless the context clearly indicates otherwise. Further, all units, prefixes, and symbols may be denoted in its SI accepted form. Numeric ranges recited within the specification are inclusive of the numbers within the defined range. Throughout this disclosure, various aspects are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range (e.g. 1 to 5 includes 1, 1.5, 2, 2.75, 3, 3.80, 4, and 5).

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments without undue experimentation. In describing and claiming the embodiments, the following terminology will be used in accordance with the definitions set out below.

The term “about”, as used herein, refers to variations in the numerical quantity that can occur, for example, through typical measuring and handling procedures; through inadvertent error in these procedures; through differences in the manufacture, source, or purity of the ingredients; and the like. Whether or not modified by the term “about”, the claims include equivalents to the quantities.

The term “alkoxy” means an-OR radical or group, where R is alkyl as defined above, e.g., methoxy, ethoxy, propoxy, or 2-propoxy, n-, iso-, or tert-butoxy, and the like. In certain embodiments, preferred alkoxy groups of the invention have 1 to 6 carbon atoms. In other embodiments, preferred alkoxy groups of the invention have three or more carbon atoms, preferably 4 to 6 carbon atoms. An alkoxy group may be optionally substituted where allowed by available valences. Examples of substituted alkoxy groups include trifluoromethoxy, hydroxymethyl, hydroxyethyl, hydroxypropyl, and alkoxyalkyl groups such as methoxymethyl, methoxyethyl, polyoxoethylene, polyoxopropylene, and similar groups. Unless specifically stated as “unsubstituted,” references to chemical moieties herein are understood to include substituted variants.

The term “alkyl” means a saturated straight chain or branched hydrocarbon chains having, for example, 1 to 20 carbon atoms. In some embodiments, the alkyl groups comprise “C1 to C6 alkyl” groups (alternatively termed “lower alkyl” groups) that include methyl, ethyl, propyl, iso-propyl n-butyl, iso-butyl, sec-butyl, t-butyl, pentyl, n-pentyl, tert-pentyl, neo-pentyl, iso-penthyl, 2-methylpentyl, 3-methylpentyl, 4-methylpentyl, 2,3-dimethylbutyl, hexyl, n-hexyl, tert-hexyl, neo-hexyl, iso-hexyl, sec-hexyl, and the like. In certain embodiments, preferred alkyl groups of the invention have 1 to 6 carbon atoms. In certain embodiments, preferred alkyl groups of the invention have three or more carbon atoms, preferably 4 to 6 carbon atoms. An alkyl group may be optionally substituted where allowed by available valences. Unless specifically stated as “unsubstituted,” references to chemical moieties herein are understood to include substituted variants.

As used herein, the term “Brix” refers to the sugar content of an aqueous solution in terms of sucrose. One degree Brix is 1 gram of sucrose in 100 grams of solution. The Brix of a sweetener compound or composition may be calculated from the degree of sweetness relative to sucrose using a sensory panel. For example, the sweetener compound of Formula (I) is about 100 times the sweetness of sucrose; therefore, the amount of the sweetener compound of Formula (I) equivalent to Brix 1 in terms of sucrose is about 100 ppm sweetener compound of Formula (I). Sweetness equivalent in Brix of sweetener compounds can be calculated similarly from the concentration response as determined using a sensory panel. Approximate relative sweetness values for sweeteners that may be used as alternatives to sucrose are listed in Table 1.1 of “Alternative Sweeteners” 4th Edition, Lyn O'Brien-Nabors CRC Press, published Oct. 26, 2016, ISBN 9781138198562-CAT#K31388. The contents of this reference are incorporated herein by reference in its entirety for all purposes.

As used herein, the terms “combination” or “combinations” refer to a mixture of two or more compounds (or other referenced components). Combinations can include, but are not limited to, a combination of one or more compounds of Formula (I) or biologically acceptable salts, derivatives, diastereomers, or enantiomers thereof, or one or more additional sweeteners.

As used herein, and unless otherwise indicated, the term “comestibly or biologically acceptable salt” refers to any comestibly or biologically acceptable salt, ester, or salt of such ester, of a compound of the present invention, which, upon ingestion, is capable of providing (directly or indirectly) a compound of the present invention, or a metabolite, residue or portion thereof, characterized by the ability to provide characteristics like that of sucrose that include maximal response, flavor profile, temporal profile, adaptation behavior and mouthfeel. In certain embodiments, preferred comestibly or biologically acceptable salts of a compound of Formula (I) are hydrochloride salts.

As used herein, the term “enantiomerically pure” means a stereomerically pure composition of a compound having at least one chiral center.

As used herein, and unless otherwise indicated, the terms “halo” and “halogen” refer to the fluoro, chloro, bromo or iodo atoms. There can be one or more halogens, which are the same or different.

As used herein, the term “pharmaceutically acceptable salt” refers to a salt prepared from a pharmaceutically acceptable non-toxic acid or base including an inorganic acid and base and an organic acid and base. Suitable pharmaceutically acceptable base addition salts of a compound provided herein (such as a compound of Formula (I), Compound 1, Compound 2, Compound 3, Compound 4, Compound 5, Compound 6, Compound 7, Compound 8 or Compound 9) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Others are well known in the art, see for example, Remington's Pharmaceutical Sciences, 18th eds., Mack Publishing, Easton Pa. (1990) or Remington: The Science and Practice of Pharmacy, 19th eds., Mack Publishing, Easton Pa. (1995).

As used herein, the term “ppm”, as used herein, means parts-per-million and is a weight relative parameter. A part-per-million is a microgram per gram, such that a component that is present at 10 ppm is present at 10 micrograms of the specific component per 1 gram of the aggregate mixture.

As used herein, the term “stereoisomer” or “stereoisomeric form” refers to one stereoisomer of a compound of Formula (I) that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

A compound of Formula (I) can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms, are encompassed by the embodiments disclosed herein. In certain embodiments racemic mixtures of isomers (about 50% by weight of each stereoisomer of the compound) are employed in the sweetener composition. These isomers may be asymmetrically synthesized or resolved using standard techniques such as chiral columns, chiral resolving agents, or chiral reduction via enzymes. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

As used herein, a compound that is “substantially pure” is substantially free from other compounds (i.e., impurities). In certain embodiments, a compound that is substantially pure contains less than about 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.05%, or 0.01% of one or more other compounds on a weight basis. The detection of other compounds can be accomplished by any method apparent to a person of ordinary skill in the art, including, but not limited to, methods of chemical analysis, such as, e.g., mass spectrometry analysis, spectroscopic analysis, thermal analysis, elemental combustion analysis and/or chromatographic analysis.

As used herein, the term “substituted” means a group may be substituted by one or more independent substituents, examples of which include, but are not limited to, halo, alkyl, alkoxy, trifluoromethyl, trifluoromethoxy, hydroxy, alkoxy, cycloalkyoxy, heterocylooxy, oxo, alkanoyl, alkylcarbonyl, cycloalkyl, aryl, aryloxy, aralkyl, alkanoyloxy, cyano, azido, amino, alkylamino, —S(O)20H, arylamino, aralkylamino, cycloalkylamino, heterocycloamino, mono and disubstituted amino in which the two substituents on the amino group are selected from alkyl, aryl, aralkyl, alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkyl sulfonyl, arylsulfonyl, aralkylsulfonyl, oxygen, sulfonamido (e.g., —SO2NH2), substituted sulfonamido, nitro, carboxy, carbamyl (e.g., —CONH2), substituted carbamyl (e.g., —CONH alkyl, —CONH aryl, —CONH aralkyl or instances where there are two substituents on the nitrogen selected from alkyl, aryl or aralkyl), alkoxycarbonyl, aryl, substituted aryl, guanidino and heterocyclo, such as indolyl, imidazolyl, furyl, thienyl, thiazolyl, pyrrolidyl, pyridyl, pyrimidyl, and the like.

As used herein, the phrases “sugar-like characteristic,” “sugar-like taste,” “sugar-like sweet,” “sugary,” and “sugar-like” are synonymous. Sugar-like characteristics include any characteristic similar to that of sucrose and include, but are not limited to, maximal response, flavor profile, temporal profile, adaptation behavior, mouthfeel, concentration/response function behavior, tastant and flavor/sweet taste interactions, and temperature effects. These characteristics are dimensions in which the taste of sucrose is different from the taste of natural and synthetic high-potency sweeteners. Whether or not a characteristic is more sugar-like is determined by expert taster or trained sensory panel assessments of sugar and compositions comprising at least one natural and/or synthetic high-potency sweetener, both with and without a sweet taste improving composition. Such assessments quantify similarities of the characteristics of compositions comprising the sweetener compositions according to the invention compared to those comprising sugar. Suitable procedures for determining whether a composition has a more sugar-like taste are well known in the art. Moreover, the flavor profile of a sweetener composition is a quantitative profile of the relative intensities of all of the taste attributes exhibited. Such profiles often are plotted as histograms or radar plots.

As used herein, “throw syrup” or “concentrated syrup” refers to a composition comprising a sweetener composition dissolved in a volume of liquid medium that is less than the volume of liquid medium contained in a finished beverage product. In one embodiment described herein, the throw syrup or concentrated syrup comprises a ratio of from 1:1 to 120:1 sweetener composition to liquid medium. The throw syrup is combined with a liquid medium to constitute the finished beverage product. The throw syrup may optionally comprise flavoring agents, coloring agents, and other additives such as food-grade acids and preservatives. The reduced volume of liquid medium in the throw syrup allows for reduced storage and shipping costs and increased shelf life. In some embodiments, the throw syrup is formulated to provide a final beverage compositions upon dilution with about a 2-fold to about a 120-fold by volume, for example, 3-fold, 4-fold, 5-fold, 6-fold, 7-fold, 8-fold, o 9-fold, 10-fold, etc. to 120-fold by volume, of a liquid medium. For example, a carbonated beverage is produced by first making concentrated syrup and then diluting the syrup with water at the time and place of making the beverage. The dilution ratio for such beverages is often 1:5, meaning one part syrup is mixed with five parts water. The beverage often is carbonated at the time of being bottled or otherwise packaged.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

The methods and compositions may comprise, consist essentially of, or consist of the components and ingredients as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions. Sweetener Compositions

In another embodiment, sweetener compositions include a sweetener compound according to Formula (I) in an effective amount to provide a sucrose sweetness equivalence of at least 1% to the sweetener composition, and at least one additional sweetener selected from a fully caloric, low calorie, and/or zero calorie sweetener. The sweetener compositions impart a more sugar-like characteristic than compositions without the sweetener compound according to Formula (I).

In some embodiments, the sweetener composition is a tabletop sweetener composition. In other embodiments, the sweetener composition is incorporated into products, such as food, beverage (non-alcoholic and alcoholic) or other consumable products. In embodiments, where the sweetener composition is a tabletop sweetener or dietary sweetener composition, it can be a zero-, low-, or mid-calorie sweetener composition.

In some embodiments, the sweetener compositions contain the sweetener compound according to Formula (I) in an effective amount to provide a sucrose sweetness equivalence of at least 1% to the sweetener composition, and the at least one additional sweetener selected from a fully caloric, low calorie, and/or zero calorie sweetener in a ratio of from about 1:10 to about 9:10, about 2:10 to about 8:10, about 3:10 to about 8:10, about 4:10 to about 8:10, or about 5:10 on a basis of sucrose sweetness equivalence of the compounds. In other embodiments the sweetener compositions contain the sweetener compound according to Formula (I) and the at least one additional sweetener in amounts of from about 25:75 to about 75:25, or about 50:50.

### Sweetener Compounds

The sweetener compositions described herein include at least one sweetener compound according to Formula (I) in addition to the at least one additional sweetener. Disclosed herein are the sweetener compounds of Formula (I):

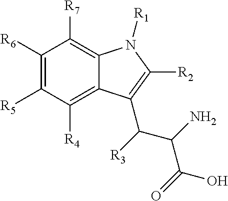

or a comestibly or biologically acceptable salt or stereoisomeric form thereof, wherein:
R1 is hydrogen or C1-C6 alkyl;
R2-R5 and R7 are each independently hydrogen, halogen, C1-C6 alkyl, C1-C6 alkoxy, hydroxyl or trifluoromethyl; and
R6 is I, hydrogen, C4-C6 alkyl, C4-C6 alkoxy or hydroxyl.

In some embodiments, an effective amount of one or more compound of Formula (I) is included in the sweetener compositions described herein to provide a sucrose sweetness equivalence of at least 1%, at least 5%, at least 10%, at least 15%, or at least 20%.

In certain embodiments, the R1, R2, R3, R5, R6, and R7 of the compounds of Formula (I) described herein are independently at each occurrence hydrogen.

In certain embodiments, the R4 of the compounds of Formula (I) described herein is methyl.

In certain embodiments, the compound of Formula (I) described herein is 2-amino-3-(4-methyl-IH-indol-3-yl)propanoic acid.

In certain embodiments, the R1, R2, R3, R4, R6, and R7 of the compounds of Formula (I) described herein are independently at each occurrence hydrogen. In certain embodiments, the R5 of the compounds of Formula (I) described herein is methyl.

In certain embodiments, the compound of Formula (I) described herein is 2-amino-3-(5-methyl-IH-indol-3-yl)propanoic acid.

In certain embodiments, the R1, R3, R4, R5, R6, and R7 of the compounds of Formula (I) described herein are independently at each occurrence hydrogen. In certain embodiments, the R2 of the compounds of Formula (I) is methyl.

In certain embodiments, the compound of Formula (I) described herein is 2-amino-3-(2-methyl-IH-indol-3-yl)propanoic acid.

In certain embodiments, the compounds of Formula (I) described herein are selected from the compounds in Table 1 or a comestibly or biologically acceptable salt or stereoisomeric form thereof. In some embodiments, the compound of Formula (I) is selected from Compound 4, Compound 5, and Compound 6. In further embodiments, the compound of Formula (I) is selected from Compound 4, Compound 5, and Compound 6, wherein the compound is the D-isomer.

TABLE 1
Compounds of Formula (I)

| Compound | Compound Strneture | Chemical Name* |
|---|---|---|
| 1 | 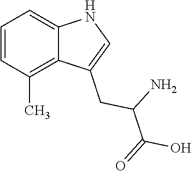 [SMILES: Cc1cccc2[nH]cc(CC(N)C(=O)O)c12] | 2-amino-3-(4- methyl-1H-indol- 3-yl)propanoic acid |
| 2 | 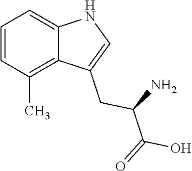 [SMILES: Cc1cccc2[nH]cc(C[C@@H](N)C(=O)O)c12] | (R)-2-amino-3-(4- methyl-1H-indol- 3-yl)propanoic acid |
| 3 | 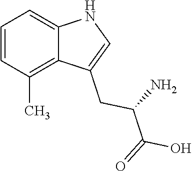 [SMILES: Cc1cccc2[nH]cc(C[C@H](N)C(=O)O)c12] | (S)-2-amino-3-(4- methyl-1H-indol- 3-yl)propanoic acid |
| 4 | 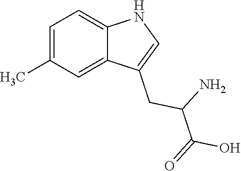 [SMILES: Cc1ccc2[nH]cc(CC(N)C(=O)O)c2c1] | 2-amino-3-(5- methyl-1H-indol- 3-yl)propanoic acid |
| 5 | 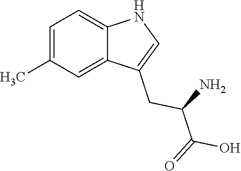 [SMILES: Cc1ccc2[nH]cc(C[C@@H](N)C(=O)O)c2c1] | (R)-2-amino-3-(5- methyl-1H-indol- 3-yl)propanoic acid |
| 6 | 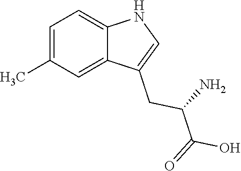 [SMILES: Cc1ccc2[nH]cc(C[C@H](N)C(=O)O)c2c1] | (S)-2-amino-3-(5- methyl-1H-indol- 3-yl)propanoic acid |
| 7 | 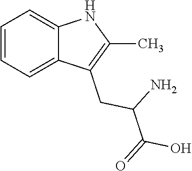 [SMILES: Cc1[nH]c2ccccc2c1CC(N)C(=O)O] | 2-amino-3-(2- methyl-1H-indol- 3-yl)propanoic acid |
| 8 | 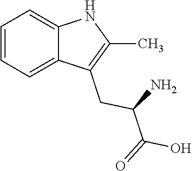 [SMILES: Cc1[nH]c2ccccc2c1C[C@@H](N)C(=O)O] | (R)-2-amino-3-(2- methyl-1H-indol- 3-yl)propanoic acid |
| 9 | 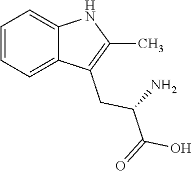 [SMILES: Cc1[nH]c2ccccc2c1C[C@H](N)C(=O)O] | (S)-2-amino-3-(2- methyl-1H-indol- 3-yl)propanoic acid |

For the purposes of this disclosure, Table 1 serves to define that a particular structure is associated with a particular name. Whenever a particular name is recited in this disclosure or the claims, the chemical structure associated with that particular name shall be the structure identified in Table 1.

In a particular embodiment, the compounds of Formula (I) provided herein are selected from: 2-amino-3-(4-methyl-1H-indol-3-yl)propanoic acid (“Compound 1”); (R)-2-amino-3-(4-methyl-1H-indol-3-yl)propanoic acid (“Compound 2”); (S)-2-amino-3-(4-methyl-1H-indol-3-yl)propanoic acid (“Compound 3”); 2-amino-3-(5-methyl-1H-indol-3-yl)propanoic acid (“Compound 4”); (R)-2-amino-3-(5-methyl-1H-indol-3-yl)propanoic acid (“Compound 5”); (S)-2-amino-3-(5-methyl-1H-indol-3-yl)propanoic acid (“Compound 6”); 2-amino-3-(2-methyl-1H-indol-3-yl)propanoic acid (“Compound 7”); (R)-2-amino-3-(2-methyl-1H-indol-3-yl)propanoic acid (“Compound 8”); and (S)-2-amino-3-(2-methyl-1H-indol-3-yl)propanoic acid (“Compound 9”); or a comestibly or biologically acceptable salt or stereoisomeric form thereof.

In some embodiments, the compounds of Formula (I) are selected from 2-amino-3-(5-methyl-1H-indol-3-yl)propanoic acid (“Compound 4”), (R)-2-amino-3-(5-methyl-1H-indol-3-yl)propanoic acid (“Compound 5”), and (S)-2-amino-3-(5-methyl-1H-indol-3-yl)propanoic acid (“Compound 6”), or a comestibly or biologically acceptable salt or stereoisomeric form thereof.

The comestibly or biologically acceptable salt of a compound provided herein includes salts derived from inorganic or organic acids and bases. Examples of such salts include, but are not limited to, those derived from appropriate bases, including alkali metal (e.g., sodium and potassium), alkaline earth metal (e.g., magnesium), ammonium and N+(Cr-4 alkyl)4 salts. In one embodiment, that salt is selected from a sodium salt, a potassium salt, an ammonium salt, or a calcium salt. In another embodiment, the salt is selected from a sodium salt, a potassium salt, or a calcium salt. In a further embodiment, the salt is a sodium salt, wherein the sodium salt of a compound provided herein is capable of disintegrating or dissolving 300-400 times more rapidly than the free base of the compound of Formula I.

In certain embodiments, a compound provided herein (such as a compound of Formula (I), Compound 1, Compound 2, Compound 3, Compound 4, Compound 5, Compound 6, Compound 7, Compound 8 or Compound 9) is isolated (including for example separated from its naturally occurring environment if it is derived from a natural source or product), synthesized, or bio-synthesized. In certain embodiments, the compound is isolated and substantially pure. In certain embodiments, the compound provided herein is no less than about 80%, no less than about 85%, no less than about 90%, no less than about 95%, no less than about 96%, no less than about 97%, no less than about 98%, no less than about 98.5%, no less than about 99%, no less than about 99.5%, or no less than about 99.8% pure.

In a particular embodiment, a compound provided herein (such as a compound of Formula (I), Compound 1, Compound 4, or Compound 7) are those wherein the compound provided herein is racemic. In a particular embodiment, the compound provided herein is the L-isomer. In a more preferred embodiment, the compound provided herein is the D-isomer. In certain embodiments, the D-isomer of a compound provided herein (such as a D-isomer of a compound of Formula (I), Compound 4, Compound 5, or Compound 6).

In the event of a discrepancy between a depicted structure and a name given that structure, the depicted structure is to be accorded more weight. Unless stated to the contrary, a formula with chemical bonds shown only as solid lines and not as wedges or dashed lines contemplates each possible isomer (e.g., each enantiomer and diastereomer, or geometric isomers (i.e., E, Z)), and a mixture of isomers, such as racemic or scalemic mixtures. Single stereochemical isomers, as well as enantiomeric and diastereomeric mixtures of a compound provided herein (such as a compound of Formula (I), Compound 1, Compound 2, Compound 3, Compound 4, Compound 5, Compound 6, Compound 7, Compound 8 or Compound 9) are within the scope of the invention. Further, unless otherwise stated, formulas depicted herein are also meant to include compounds which differ only in the presence of one or more isotopically enriched atoms. For example, Formula (I) compounds provided herein having the present formulas except for the replacement of a hydrogen by a deuterium or tritium, or the replacement of a carbon by a 13C— or 14C— enriched carbon are within the scope of this invention.

The compounds provided herein (such as a compound of Formula (I), Compound 1, Compound 2, Compound 3, Compound 4, Compound 5, Compound 6, Compound 7, Compound 8 or Compound 9) can also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

In one embodiment, one or more Formula (I) compounds described herein is contained in an aqueous solution in an effective amount to impart a maximum sweetness intensity equivalent to that of a 20%, 25%, 30%, 35%, or 40% aqueous solution of sucrose by weight. In another embodiment, about 50 mg-100 mg of one or more Formula (I) compounds described herein in 100 mg of water is equivalent to 1 degree Brix. In another embodiment, one or more Formula (I) compounds described herein provides from about 1 to about 12 degrees Brix when added to an unsweetened or sweetened beverage product (based on the amount of compounds provided).

Additional disclosure of the Formula (I) compounds described herein is set forth in PCT Filing No. PCT/US18/064473, which claims priority to U.S. Ser. No. 62/596,667, both of which are herein incorporated by reference in their entirety.

### Additional Sweeteners

The sweetener compositions include at least one additional sweetener in addition to the sweetener compound according to Formula (I). Suitable sweeteners for combining with the sweetener compound include for example, fully caloric, low calorie, and/or zero calorie sweeteners. These sweeteners can also be referred to, respectively, as nutritive sweeteners, partially-nutritive sweeteners, and non-nutritive sweeteners (i.e. high potency sweeteners (or high intensity sweeteners)). Non-nutritive sweeteners including both natural and synthetic sweeteners (i.e. artificial sweeteners) and along with partially-nutritive sweeteners are an alternative to nutritive sweeteners to provide desirable taste characteristics as well as other functional properties with significantly lower caloric content. Non-nutritive sweeteners do not contribute to the caloric content of sweetener compositions.

Exemplary zero calorie sweeteners (i.e. non-nutritive, natural high-potency sweeteners) include sweeteners found in nature which may be in raw, extracted, purified, or any other form (e.g. via fermentation, bio-conversion), singularly or in combination thereof and characteristically have a sweetness potency greater than sucrose, fructose, or glucose, whiling having less calories. Non-limiting examples of natural zero calorie sweeteners include steviol glycosides, including rebaudioside A (Reb A), rebaudioside B (Reb B), rebaudioside C (Reb C), rebaudioside D (Reb D), rebaudioside D2 (Reb D2), rebaudioside D4 (Reb D4), rebaudioside E (Reb E), rebaudioside F (Reb F), rebaudioside G (Reb G), rebaudioside H (Reb H), rebaudioside I (Reb I), rebaudioside J (Reb J), rebaudioside K (Reb K), rebaudioside L (Reb L), rebaudioside M2 (Reb M2), rebaudioside M (Reb M) (also known as REB X), rebaudioside N (Reb N), rebaudioside O (Reb O), rebaudioside S (Reb S), rebaudioside T (Reb T), rebaudioside U (Reb U), rebaudioside V (Reb V), rebaudioside W (Reb W), rebaudioside Z1 (Reb Z1), rebaudioside Z2 (Reb Z2), and enzymatically glucosylated steviol glycosides; amino acids, tryptophans, steviolmonoside, steviolbioside, dulcoside A, dulcoside B, rubusoside, stevia, stevioside, mogroside, mogroside IV, mogroside V, mogroside VI, iso-mogroside V, grosmomoside, neomogroside, siamenoside, Luo Han Guo sweetener, monk fruit, siamenoside, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobtain, baiyunoside, osladin, polypodoside A, pterocaryoside A, pterocaryoside B, mukurozioside, phlomisoside I, periandrin I, abrusoside A, and cyclocarioside I. Natural high-potency sweeteners also include modified natural high-potency sweeteners.

Exemplary amino acid additives include, but are not limited to, aspartic acid, arginine, glycine, glutamic acid, praline, threonine, theanine, cysteine, cystine, alanine, valine, tyrosine, leucine, arabinose, trans-4-hydroxyproline, isoleucine, asparagine, serine, lysine, histidine, omithine, methionine, carnitine, aminobutyric acid (α-, (β-, and/or δ-isomers), glutamine, hydroxyproline, taurine, norvaline, sarcosine, and their salt forms such as sodium or potassium salts or acid salts. The amino acid additives also may be in the D- or L-configuration. The amino acids may be natural or synthetic. The amino acids also may be modified. Modified amino acids refers to any amino acid wherein at least one atom has been added, removed, substituted, or combinations thereof (e.g., N-alkyl amino acid, N-acyl amino acid, or N-methyl amino acid). As used herein, amino acids also encompass both peptides and polypeptides (e.g., di peptides, tripeptides, tetrapeptides, and pentapeptides) such as glutathione and L-alanyl-L-glutamine.

Exemplary polyamino acid additives include poly-L-aspartic acid, poly-L-lysine (e.g. poly-L-α-lysine or poly-L-ε-lysine), poly-L-omithine (e.g. poly-L-α-omithine or poly-L-ε-ornithine), poly-L-arginine, other polymeric forms of amino acids, and salt forms thereof (e.g. calcium, potassium, sodium, or magnesium salts such as L-glutamic acid mono sodium salt). Suitable sugar acid additives include, but are not limited to, aldonic, uronic, aldaric, alginic, gluconic, glucuronic, glucaric, galactaric, galacturonic, and salts thereof (e.g. sodium, potassium, calcium, magnesium salts or other physiologically acceptable salts), and combinations thereof. Suitable nucleotide additives include, but are not limited to, inosine monophosphate (“IMP”), guanosine monophosphate (“GMP”), adenosine monophosphate (“AMP”), cytosine monophosphate (CMP), uracil monophosphate (UMP), inosine diphosphate, guanosine diphosphate, adenosine diphosphate, cytosine diphosphate, uracil diphosphate, inosine triphosphate, guanosine triphosphate, adenosine triphosphate, cytosine triphosphate, uracil triphosphate, alkali or alkaline earth metal salts thereof, and combinations thereof. The nucleotides described herein also may comprise nucleotide-related additives, such as nucleosides or nucleic acid bases (e.g. guanine, cytosine, adenine, thymine, uracil). Exemplary non-nutritive, synthetic high-potency sweeteners include those not found naturally in nature and characteristically having a sweetness potency greater than sucrose, fructose, or glucose, yet have fewer or no calories.

Further exemplary synthetic zero calorie (i.e. high-potency) sweeteners include sucralose, potassium acesulfame (Acesulfame-potassium), aspartame, alitame, saccharin, neohesperidin dihydrochalcone, cyclamate, neotame, advantame, N—[N-[3-(3-hydroxy-4-methoxyphenyl)propyl]-L-a-aspartyl]-L-phenylalanine I-methyl ester, N—[N-[3-(3-hydroxy-4-methoxyphenyl)-3-methylbutyl]-L-a-aspartyl]-L-phenylalanine I-methyl ester, N—[N-[3-(3-methoxy-4-hydroxyphenyl)propyl]L-a-aspartyl]-L-phenylalanine I-methyl ester, salts thereof and the like. Synthetic high-potency sweeteners also include modified synthetic high-potency sweeteners.

Low calorie (i.e. partially-nutritive) sweeteners contribute to the calorie content of the composition. In some embodiments, the use of a low calorie sweetener may provide compositions that are “mid-calorie”, such that they impart the desired sweetness when added to a sweetenable composition (such as, for example, as beverage product) and have less than about 60 calories per 8 oz serving. In other embodiments, the use of a low calorie sweetener may provide compositions that are “low-calorie”, such that they impart the desired sweetness when added to a sweetenable composition (such as, for example, a beverage product) and have less than 40 calories per 8 oz serving.

Exemplary low calorie sweeteners include polylols. The term “polyol”, as used herein, refers to a molecule that contains more than one hydroxyl group. A polyol may be a diol, triol, or a tetraol which contains 2, 3, and 4 hydroxyl groups respectively. A polyol also may contain more than 4 hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, and sugar alcohols or any other carbohydrates capable of being reduced which do not adversely affect the taste of the sweetened compositions.

Additional exemplary low calorie sweeteners include allulose (also referred to as D-allulose, psicose, D-psicose). In some embodiments, allulose includes mixtures of allulose and additional monosaccharides and disaccharides, determined according to the purity level of the allulose.

In some embodiments, fully caloric sweeteners can be included as nutritive sweeteners and often include carbohydrate sweeteners. Examples of carbohydrate sweeteners include, but are not limited to, sucrose, cane sugar, fructose, glucose, maple syrup, honey, molasses, erythritol, maltitol, lactitol, sorbitol, mannitol, xylitol, D-psicose, D-tagatose, leucrose, trehalose, galactose, rhamnose, cyclodextrin (e.g., a-cyclodextrin, P-cyclodextrin, and y-cyclodextrin), ribulose, threose, arabinose, xylose, lyxose, allose, altrose, mannose, idose, lactose, maltose, invert sugar, isotrehalose, neotrehalose, palatinose or isomaltulose, erythrose, deoxyribose, gulose, idose, talose, erythrulose, xylulose, psicose, turanose, cellobiose, glucosamine, mannosamine, fucose, fuculose, glucuronic acid, gluconic acid, glucono-lactone, abequose, galactosamine, xylo-oligosaccharides (xylotriose, xylobiose and the like), gentio-oligoscaccharides (gentiobiose, gentiotriose, gentiotetraose and the like), galacto-oligosaccharides, sorbose, ketotriose (dehydroxyacetone), aldotriose (glyceraldehyde), nigero-oligosaccharides, fructooligosaccharides (kestose, nystose and the like), maltotetraose, maltotriol, tetrasaccharides, mannan-oligosaccharides, malto-oligosaccharides (maltotriose, maltotetraose, maltopentaose, maltohexaose, maltoheptaose and the like), dextrine, lactulose, melibiose, rhamnose, ribose, isomerized liquid sugars such as high fructose corn/starch syrup (HFCS/HFSS) (e.g., HFCS55, HFCS42, or HFCS90), coupling sugars, soybean oligosaccharides, glucose syrup and combinations thereof.

In another embodiment, the additional sweetener is selected from Reb A, Reb B, Reb D, Reb M, Reb J, stevioside, mogroside, erythritol, allulose, HFCS, sucrose, cane sugar, aspartame, acesulfame-potassium, sucralose, and any combination of two or more of the foregoing.

In another embodiment, the sweetener composition comprises the sweetener compound of Formula (I) and Reb A as the additional sweetener component. The relative weight percent of the sweetener compound of Formula (I) and Reb A can each vary from about 1% to about 99%, such as for example, about 95% compound of Formula (I)/5% Reb A, about 90% compound of Formula (I)/10% Reb A, about 85% compound of Formula (I)/15% Reb A, about 80% compound of Formula (I)/20% Reb A, about 75% compound of Formula (I)/25% Reb A, about 70% compound of Formula (I)/30% Reb A, about 65% compound of Formula (I)/35% Reb A, about 60% compound of Formula (I)/40% Reb A, about 55% compound of Formula (I)/45% Reb A, about 50% compound of Formula (I)/50% Reb A, about 45% compound of Formula (I)/55% Reb A, about 40% compound of Formula (I)/60% Reb A, about 35% compound of Formula (I)/65% Reb A, about 30% compound of Formula (I)/70% Reb A, about 25% compound of Formula (I)/75% Reb A, about 20% compound of Formula (I)/80% Reb A, about 15% compound of Formula (I)/85% Reb A, about 10% compound of Formula (I)/90% Reb A or about 5% compound of Formula (I)/10% Reb A, inclusive of ranges therein.

In yet another embodiment, the sweetener composition comprises the sweetener compound of Formula (I) and Reb B as the additional sweetener component. The relative weight percent of the sweetener compound of Formula (I) and Reb B can each vary from about 1% to about 99%, such as for example, about 95% compound of Formula (I)/5% Reb B, about 90% compound of Formula (I)/10% Reb B, about 85% compound of Formula (I)/15% Reb B, about 80% compound of Formula (I)/20% Reb B, about 75% compound of Formula (I)/25% Reb B, about 70% compound of Formula (I)/30% Reb B, about 65% compound of Formula (I)/35% Reb B, about 60% compound of Formula (I)/40% Reb B, about 55% compound of Formula (I)/45% Reb B, about 50% compound of Formula (I)/50% Reb B, about 45% compound of Formula (I)/55% Reb B, about 40% compound of Formula (I)/60% Reb B, about 35% compound of Formula (I)/65% Reb B, about 30% compound of Formula (I)/70% Reb B, about 25% compound of Formula (I)/75% Reb B, about 20% compound of Formula (I)/80% Reb B, about 15% compound of Formula (I)/85% Reb B, about 10% compound of Formula (I)/90% Reb B or about 5% compound of Formula (I)/10% Reb B, inclusive of ranges therein.

In still another embodiment, the sweetener composition comprises the sweetener compound of Formula (I) and Reb D as the additional sweetener component. The relative weight percent of the sweetener compound of Formula (I) and Reb D can each vary from about 1% to about 99%, such as for example, about 95% compound of Formula (I)/5% Reb D, about 90% compound of Formula (I)/10% Reb D, about 85% compound of Formula (I)/15% Reb D, about 80% compound of Formula (I)/20% Reb D, about 75% compound of Formula (I)/25% Reb D, about 70% compound of Formula (I)/30% Reb D, about 65% compound of Formula (I)/35% Reb D, about 60% compound of Formula (I)/40% Reb D, about 55% compound of Formula (I)/45% Reb D, about 50% compound of Formula (I)/50% Reb D, about 45% compound of Formula (I)/55% Reb D, about 40% compound of Formula (I)/60% Reb D, about 35% compound of Formula (I)/65% Reb D, about 30% compound of Formula (I)/70% Reb D, about 25% compound of Formula (I)/75% Reb D, about 20% compound of Formula (I)/80% Reb D, about 15% compound of Formula (I)/85% Reb D, about 10% compound of Formula (I)/90% Reb D or about 5% compound of Formula (I)/10% Reb D, inclusive of ranges therein.

In even yet another embodiment, the sweetener composition comprises the sweetener compound of Formula (I) and Reb M as the additional sweetener component. The relative weight percent of the sweetener compound of Formula (I) and Reb M can each vary from about 1% to about 99%, such as for example, about 95% compound of Formula (I)/5% Reb M, about 90% compound of Formula (I)/10% Reb M, about 85% compound of Formula (I)/15% Reb M, about 80% compound of Formula (I)/20% Reb M, about 75% compound of Formula (I)/25% Reb M, about 70% compound of Formula (I)/30% Reb M, about 65% compound of Formula (I)/35% Reb M, about 60% compound of Formula (I)/40% Reb M, about 55% compound of Formula (I)/45% Reb M, about 50% compound of Formula (I)/50% Reb M, about 45% compound of Formula (I)/55% Reb M, about 40% compound of Formula (I)/60% Reb M, about 35% compound of Formula (I)/65% Reb M, about 30% compound of Formula (I)/70% Reb M, about 25% compound of Formula (I)/75% Reb M, about 20% compound of Formula (I)/80% Reb M, about 15% compound of Formula (I)/85% Reb M, about 10% compound of Formula (I)/90% Reb M or about 5% compound of Formula (I)/10% Reb M, inclusive of ranges therein.

In still another embodiment, the sweetener composition comprises the sweetener compound of Formula (I) and stevioside as the additional sweetener component. The relative weight percent of the sweetener compound of Formula (I) and stevioside can each vary from about 1% to about 99%, such as for example, about 95% compound of Formula (I)/5% stevioside, about 90% compound of Formula (I)/10% stevioside, about 85% compound of Formula (I)/15% stevioside, about 80% compound of Formula (I)/20% stevioside, about 75% compound of Formula (I)/25% stevioside, about 70% compound of Formula (I)/30% stevioside, about 65% compound of Formula (I)/35% stevioside, about 60% compound of Formula (I)/40% stevioside, about 55% compound of Formula (I)/45% stevioside, about 50% compound of Formula (I)/50% stevioside, about 45% compound of Formula (I)/55% stevioside, about 40% compound of Formula (I)/60% stevioside, about 35% compound of Formula (I)/65% stevioside, about 30% compound of Formula (I)/70% stevioside, about 25% compound of Formula (I)/75% stevioside, about 20% compound of Formula (I)/80% stevioside, about 15% compound of Formula (I)/85% stevioside, about 10% compound of Formula (I)/90% stevioside or about 5% compound of Formula (I)/10% stevioside, inclusive of ranges therein.

In still another embodiment, the sweetener composition comprises the sweetener compound of Formula (I) and mogroside as the additional sweetener component. The relative weight percent of the sweetener compound of Formula (I) and mogroside can each vary from about 1% to about 99%, such as for example, about 95% compound of Formula (I)/5% mogroside, about 90% compound of Formula (I)/10% mogroside, about 85% compound of Formula (I)/15% mogroside, about 80% compound of Formula (I)/20% mogroside, about 75% compound of Formula (I)/25% mogroside, about 70% compound of Formula (I)/30% mogroside, about 65% compound of Formula (I)/35% mogroside, about 60% compound of Formula (I)/40% mogroside, about 55% compound of Formula (I)/45% mogroside, about 50% compound of Formula (I)/50% mogroside, about 45% compound of Formula (I)/55% mogroside, about 40% compound of Formula (I)/60% mogroside, about 35% compound of Formula (I)/65% mogroside, about 30% compound of Formula (I)/70% mogroside, about 25% compound of Formula (I)/75% mogroside, about 20% compound of Formula (I)/80% mogroside, about 15% compound of Formula (I)/85% mogroside, about 10% compound of Formula (I)/90% mogroside or about 5% compound of Formula (I)/10% mogroside, inclusive of ranges therein.

In another embodiment, the sweetener composition comprises the sweetener compound of Formula (I) and allulose as the additional sweetener component. The relative weight percent of the sweetener compound of Formula (I) and allulose can each vary from about 1% to about 99%, such as for example, about 95% compound of Formula (I)/5% allulose, about 90% compound of Formula (I)/10% allulose, about 85% compound of Formula (I)/15% allulose, about 80% compound of Formula (I)/20% allulose, about 75% compound of Formula (I)/25% allulose, about 70% compound of Formula (I)/30% allulose, about 65% compound of Formula (I)/35% allulose, about 60% compound of Formula (I)/40% allulose, about 55% compound of Formula (I)/45% allulose, about 50% compound of Formula (I)/50% allulose, about 45% compound of Formula (I)/55% allulose, about 40% compound of Formula (I)/60% allulose, about 35% compound of Formula (I)/65% allulose, about 30% compound of Formula (I)/70% allulose, about 25% compound of Formula (I)/75% allulose, about 20% compound of Formula (I)/80% allulose, about 15% compound of Formula (I)/85% allulose, about 10% compound of Formula (I)/90% allulose or about 5% compound of Formula (I)/10% allulose, inclusive of ranges therein.

In still yet another embodiment, the sweetener composition comprises the sweetener compound of Formula (I) and aspartame as the additional sweetener component. The relative weight percent of the sweetener compound of Formula (I) and aspartame can each vary from about 1% to about 99%, such as for example, about 95% compound of Formula (I)/5% aspartame, about 90% compound of Formula (I)/10% aspartame, about 85% compound of Formula (I)/15% aspartame, about 80% compound of Formula (I)/20% aspartame, about 75% compound of Formula (I)/25% aspartame, about 70% compound of Formula (I)/30% aspartame, about 65% compound of Formula (I)/35% aspartame, about 60% compound of Formula (I)/40% aspartame, about 55% compound of Formula (I)/45% aspartame, about 50% compound of Formula (I)/50% aspartame, about 45% compound of Formula (I)/55% aspartame, about 40% compound of Formula (I)/60% aspartame, about 35% compound of Formula (I)/65% aspartame, about 30% compound of Formula (I)/70% aspartame, about 25% compound of Formula (I)/75% aspartame, about 20% compound of Formula (I)/80% aspartame, about 15% compound of Formula (I)/85% aspartame, about 10% compound of Formula (I)/90% aspartame or about 5% compound of Formula (I)/10% aspartame, inclusive of ranges therein.

In another embodiment, the sweetener composition comprises the sweetener compound of Formula (I) and acesulfame-potassium as the additional sweetener component. The relative weight percent of the sweetener compound of Formula (I) and acesulfame-potassium can each vary from about 1% to about 99%, such as for example, about 95% compound of Formula (I)/5% acesulfame-potassium, about 90% compound of Formula (I)/10% acesulfame-potassium, about 85% compound of Formula (I)/15% acesulfame-potassium, about 80% compound of Formula (I)/20% acesulfame-potassium, about 75% compound of Formula (I)/25% acesulfame-potassium, about 70% compound of Formula (I)/30% acesulfame-potassium, about 65% compound of Formula (I)/35% acesulfame-potassium, about 60% compound of Formula (I)/40% acesulfame-potassium, about 55% compound of Formula (I)/45% acesulfame-potassium, about 50% compound of Formula (I)/50% acesulfame-potassium, about 45% compound of Formula (I)/55% acesulfame-potassium, about 40% compound of Formula (I)/60% acesulfame-potassium, about 35% compound of Formula (I)/65% acesulfame-potassium, about 30% compound of Formula (I)/70% acesulfame-potassium, about 25% compound of Formula (I)/75% acesulfame-potassium, about 20% compound of Formula (I)/80% acesulfame-potassium, about 15% compound of Formula (I)/85% acesulfame-potassium, about 10% compound of Formula (I)/90% acesulfame-potassium or about 5% compound of Formula (I)/10% acesulfame-potassium, inclusive of ranges therein.

In an embodiment, the sweetener composition comprises the sweetener compound of Formula (I) and sucralose as the additional sweetener component. The relative weight percent of the sweetener compound of Formula (I) and sucralose can each vary from about 1% to about 99%, such as for example, about 95% compound of Formula (I)/5% sucralose, about 90% compound of Formula (I)/10% sucralose, about 85% compound of Formula (I)/15% sucralose, about 80% compound of Formula (I)/20% sucralose, about 75% compound of Formula (I)/25% sucralose, about 70% compound of Formula (I)/30% sucralose, about 65% compound of Formula (I)/35% sucralose, about 60% compound of Formula (I)/40% sucralose, about 55% compound of Formula (I)/45% sucralose, about 50% compound of Formula (I)/50% sucralose, about 45% compound of Formula (I)/55% sucralose, about 40% compound of Formula (I)/60% sucralose, about 35% compound of Formula (I)/65% sucralose, about 30% compound of Formula (I)/70% sucralose, about 25% compound of Formula (I)/75% sucralose, about 20% compound of Formula (I)/80% sucralose, about 15% compound of Formula (I)/85% sucralose, about 10% compound of Formula (I)/90% sucralose or about 5% compound of Formula (I)/10% sucralose, inclusive of ranges therein.

In even still yet another embodiment, the sweetener composition comprises the sweetener compound of Formula (I) and erythritol as the additional sweetener component. The relative weight percent of the sweetener compound of Formula (I) and erythritol can each vary from about 1% to about 99%, such as for example, about 95% compound of Formula (I)/5% erythritol, about 90% compound of Formula (I)/10% erythritol, about 85% compound of Formula (I)/15% erythritol, about 80% compound of Formula (I)/20% erythritol, about 75% compound of Formula (I)/25% erythritol, about 70% compound of Formula (I)/30% erythritol, about 65% compound of Formula (I)/35% erythritol, about 60% compound of Formula (I)/40% erythritol, about 55% compound of Formula (I)/45% erythritol, about 50% compound of Formula (I)/50% erythritol, about 45% compound of Formula (I)/55% erythritol, about 40% compound of Formula (I)/60% erythritol, about 35% compound of Formula (I)/65% erythritol, about 30% compound of Formula (I)/70% erythritol, about 25% compound of Formula (I)/75% erythritol, about 20% compound of Formula (I)/80% erythritol, about 15% compound of Formula (I)/85% erythritol, about 10% compound of Formula (I)/90% erythritol or about 5% compound of Formula (I)/10% erythritol, inclusive of ranges therein.

In a still further embodiment, the sweetener composition comprises the sweetener compound of Formula (I) and HFCS as the additional sweetener component. The relative weight percent of the sweetener compound of Formula (I) and HFCS can each vary from about 1% to about 99%, such as for example, about 95% compound of Formula (I)/5% HFCS, about 90% compound of Formula (I)/10% HFCS, about 85% compound of Formula (I)/15% HFCS, about 80% compound of Formula (I)/20% HFCS, about 75% compound of Formula (I)/25% HFCS, about 70% compound of Formula (I)/30% HFCS, about 65% compound of Formula (I)/35% HFCS, about 60% compound of Formula (I)/40% HFCS, about 55% compound of Formula (I)/45% HFCS, about 50% compound of Formula (I)/50% HFCS, about 45% compound of Formula (I)/55% HFCS, about 40% compound of Formula (I)/60% HFCS, about 35% compound of Formula (I)/65% HFCS, about 30% compound of Formula (I)/70% HFCS, about 25% compound of Formula (I)/75% HFCS, about 20% compound of Formula (I)/80% HFCS, about 15% compound of Formula (I)/85% HFCS, about 10% compound of Formula (I)/90% HFCS or about 5% compound of Formula (I)/10% HFCS, inclusive of ranges therein.

In an even still further embodiment, the sweetener composition comprises the sweetener compound of Formula (I) and sucrose as the additional sweetener component. The relative weight percent of the sweetener compound of Formula (I) and sucrose can each vary from about 1% to about 99%, such as for example, about 95% compound of Formula (I)/5% sucrose, about 90% compound of Formula (I)/10% sucrose, about 85% compound of Formula (I)/15% sucrose, about 80% compound of Formula (I)/20% sucrose, about 75% compound of Formula (I)/25% sucrose, about 70% compound of Formula (I)/30% sucrose, about 65% compound of Formula (I)/35% sucrose, about 60% compound of Formula (I)/40% sucrose, about 55% compound of Formula (I)/45% sucrose, about 50% compound of Formula (I)/50% sucrose, about 45% compound of Formula (I)/55% sucrose, about 40% compound of Formula (I)/60% sucrose, about 35% compound of Formula (I)/65% sucrose, about 30% compound of Formula (I)/70% sucrose, about 25% compound of Formula (I)/75% sucrose, about 20% compound of Formula (I)/80% sucrose, about 15% compound of Formula (I)/85% sucrose, about 10% compound of Formula (I)/90% sucrose or about 5% compound of Formula (I)/10% sucrose, inclusive of ranges therein.

In another embodiment, the sweetener composition comprises the sweetener compound of Formula (I) and cane sugar as the additional sweetener component. The relative weight percent of the sweetener compound of Formula (I) and cane sugar can each vary from about 1% to about 99%, such as for example, about 95% compound of Formula (I)/5% cane sugar, about 90% compound of Formula (I)/10% cane sugar, about 85% compound of Formula (I)/15% cane sugar, about 80% compound of Formula (I)/20% cane sugar, about 75% compound of Formula (I)/25% cane sugar, about 70% compound of Formula (I)/30% cane sugar, about 65% compound of Formula (I)/35% cane sugar, about 60% compound of Formula (I)/40% cane sugar, about 55% compound of Formula (I)/45% cane sugar, about 50% compound of Formula (I)/50% cane sugar, about 45% compound of Formula (I)/55% cane sugar, about 40% compound of Formula (I)/60% cane sugar, about 35% compound of Formula (I)/65% cane sugar, about 30% compound of Formula (I)/70% cane sugar, about 25% compound of Formula (I)/75% cane sugar, about 20% compound of Formula (I)/80% cane sugar, about 15% compound of Formula (I)/85% cane sugar, about 10% compound of Formula (I)/90% cane sugar or about 5% compound of Formula (I)/10% cane sugar, inclusive of ranges therein.

Food, Beverage, and/or Other Consumable Products Containing One or More Sweetener Composition Described Herein

One or more sweetener composition described herein is suitable for use in diverse applications, including but not limited to food products, beverage products, and/or other consumable products. Among the advantages of the sweetener compositions is that they may be readily incorporated into known processes for making foods, beverages, and other consumable products without any additional processing steps. For example, the sweetener compositions described herein may be mixed with a solvent at room temperature or under heat (e.g. gradient heat treatment) before or when being combined with other ingredients of food, beverage, or other consumable products.

In exemplary embodiments, the sweetener compositions can be included in beverage product compositions in amounts between about 5 ppm to about 4000 ppm, about 5 ppm to about 50 ppm, about 10 ppm to about 100 ppm, about 20 ppm to about 100 ppm; about 20 pm to about 200 ppm; about 20 ppm to about 300 ppm; about 20 ppm to about 400 pppm; about 20 ppm to about 500 ppm; about 50 ppm to about 500 ppm; about 100 ppm to about 500 ppm; about 100 ppm to about 700 ppm; about 100 ppm to about 800 ppm; about 100 ppm to about 900 ppm; about 100 ppm to about 1000 ppm; about 500 ppm to about 1000 ppm; about 500 ppm to about 1100 ppm; about 500 ppm to about 1200 ppm; about 500 ppm to about 1300 ppm; about 500 ppm to about 1400 ppm; about 500 ppm to about 1500 ppm; about 1000 ppm to about 1600 ppm; about 1000 ppm to about 1700 ppm; about 1500 ppm to about 1800 ppm; about 1500 ppm to about 1900 ppm; about 1500 ppm to about 2000 ppm, about 2000 ppm to about 2100 ppm; about 2100 ppm to about 2200 ppm; about 2200 ppm to about 2300 ppm; about 2300 ppm to about 2400 ppm; about 2400 ppm to about 2500 ppm; about 2500 ppm to about 2600 ppm; about 2600 ppm to about 2700 ppm; about 2700 ppm to about 2800 ppm; about 2800 ppm to about 2900 ppm, about 2900 ppm to about 3000 ppm, about 3000 ppm to about 3100 ppm, about 3100 ppm to about 3200 ppm, about 3200 ppm to about 3300 ppm, about 3300 ppm to about 3400 ppm, about 3400 ppm to about 3500 ppm, about 3500 ppm to about 3600 ppm, about 3600 ppm to about 3700 ppm, about 3700 ppm to about 3800 ppm, about 3800 ppm to about 3900, or about 3900 to about 4000 ppm, or increments in between those recited.

In some embodiments, one or more beverage product described herein is a mid-calorie, low-calorie or zero-calorie, non-alcoholic and alcoholic beverage.

In exemplary embodiments, the sweetener compositions can be included in food product compositions in amounts between about 0.1 wt-% to about 50 wt-%, about 0.5 wt-% to about 40 wt-%, about 1 wt-% to about 40 wt-%, about 5 wt-% to about 35 wt-%, about 5 wt-% to about 30 wt-%, about 10 wt-% to about 30 wt-%, about 10 wt-% to about 25 wt-%, about 10 wt-% to about 20 wt-%, or increments in between those recited.

The disclosed concentrations of the sweetener compound of Formula (I) in the food, beverage, and/or other consumable products beneficially provides a desirable degree of a sugar-like characteristic to such food, beverage, and/or other consumable product composition, wherein the sugar-like characteristic can include maximal response, flavor profile, temporal profile, adaptation behavior, mouthfeel, concentration/response function behavior, tastant and flavor/sweet taste interactions, and temperature effects. In other embodiments, the sweetener compositions suppress, reduce or eliminate at least one undesirable taste associated with sweeteners, wherein the at least one undesirable taste can include delayed sweetness onset, lingering sweet aftertaste, metallic taste, bitter taste, cooling sensation taste, menthol-like taste, or licorice-like taste.

Exemplary food, beverage, and/or other consumable food product compositions containing the sweetener compound of Formula (I) are set forth in the following Table 2. The exemplary food, beverage, and/or other consumable food product compositions provide ppm ranges for the typical concentrations of one or more Formula I compound when used as a single sweetener.

Concentrations may vary depending upon formulation, flavor, and target consumer.

TABLE 2
Exemplary Consumable Products Containing the Sweetener
Compound of Formula (I)

| Applications | Range ppm (mg/l) | SSE (final) |
|---|---|---|
| Juice and Juice beverages | 150-1200 | 1.5-12.0 |
| CSDS | 150-1500 | 1.5-15.0 |
| Energy drinks | 150-1500 | 1.5-15.0 |
| RTD tea & coffee | 150-1500 | 1.5-15.0 |
| Flavored water | 150-1000 | 1.5-10.0 |
| Dairy (including frozen | 1.5-20.0 | |
| dairy (e.g. ice cream) and | 50-2000 | |
| cheese) | | |
| Alternative Dairy | 150-2000 | 1.5-20 |
| Powdered beverages | 400-4000 | |
| Table top | 600-10,000 | |
| Cereal | 200-2000 | 2-20 |
| Confections (including | 200-2000 | 2-20 |
| jams and jellies) | | |
| Pharmaceuticals | 150-2000 | 1.5-20 |
| Chewing gum | 300-4000 | |
| Protein beverages | 150-1500 | 1.5-15.0 |
| Dressings | 150-1000 | 1.5-10.0 |
| Sauces/Gravy | 150-1000 | 1.5-10.0 |
| Desserts (including | 150-2000 | 1.5-20.0 |
| frozen) | | |
| Frozen foods | 150-4000 | |
| Fruit prep/concentrates | 150-12,000 | |
| Canned Food (including | | 1.5-20 |
| processed fruits & | 150-2000 | |
| vegetables) | | |
| Snacks | 150-1500 | 1.5-15 |
| Bars | 150-1500 | 1.5-15 |
| Dried blends (K pods etc.) | 150-4000 | |
| Bakery products | 150-2000 | 1.5-20 |
| 5-1 Throw syrup for | 900-6000 | |
| CSDS, Energy drinks | | |
| 9-1Throw syrup Coffee | 1500-10,000 | |
| Water Enhancers (120 | 18,000-120,000 | |
| times) | | |

Examples of food products include, but are not limited to, confections, condiments, chewing gum, frozen foods, desserts, canned foods, soy-based products, dressings, mousse, bars, snacks, whipped topping, mayonnaise, sauces, gravy, spreads, vinegar, ice cream, cereal compositions, baked goods, dairy products such as yogurts, fruit preparations anc concentrates, nondairy products, protein products, snacks and tabletop sweetener compositions.

Examples of beverage products include, but are not limited to, ready-to-drink products that are carbonated (e.g., colas or other soft drinks, sparkling beverages, alcoholic beverages and malts) or non-carbonated (e.g., fruit juices, fruit containing beverages, nectars, vegetable juices, sports drinks, energy drinks, enhanced water, coconut waters teas, RTD teas, non dairy beverages, protein beverages, meal replacement beverages, coffees, cocoa drinks, beverages containing milk, coffee creamers, beverages containing cereal extracts, smoothies, and alcoholic beverages), as well as powdered beverage products (dry mixes) that are to be combined with a liquid base such as water, milk, or club soda and beverage concentrates such as throw syrups. Throw syrups can be used to prepare any of the beverages described herein by adding an appropriate amount of water or other liquid medium.

In some embodiments, the beverage products and throw syrups disclosed herein may include a juice-based composition obtained from fruit or vegetable. The juice-based composition can be used in any form such as a juice form, a concentrate, an extract, a powder (which can be reconstituted with water or other suitable liquids), or the like. Suitable juices include, for example, non-citrus juices such as apple juice, grape juice, pear juice, nectarine juice, currant juice, raspberry juice, gooseberry juice, blackberry juice, blueberry juice, strawberry juice, custard-apple juice, pomegranate juice, guava juice, kiwi juice, mango juice, papaya juice, watermelon juice, cantaloupe juice, cherry juice, cranberry juice, peach juice, apricot juice, plum juice, and pineapple juice; citrus juices such as orange juice, lemon juice, lime juice, grapefruit juice, and tangerine juice; and vegetable juice such as carrot juice and tomato juice; or any combination thereof. The beverage products and throw syrups disclosed herein may comprise fruit or vegetable liquids containing a percentage of solids derived from the fruit or vegetable, for example pulp, seeds, skins, fibers, and the like, and pectin, which is naturally occurring in the fruit or vegetable. In some embodiments, the juice-based composition is fortified with solubilized calcium in the form of calcium carbonate, calcium lactate, calcium oxide, or calcium hydroxide.

In some embodiments, the beverage products and throw syrups disclosed herein comprise a dairy composition, wherein the dairy composition contains a dairy protein. Exemplary dairy compositions include any type of dairy product including cream, whole milk, reduced fat milk, skim milk, milk solids, condensed milk, or any combination thereof, specifically a combination of cream and skim milk. The dairy composition generally comprises an amount of dairy protein, for example whey protein containing beta-lactoglobulin, alpha-lactalbumin, or serum albumin; and the like. In some embodiments, the dairy product may be replaced with an amount of a non-dairy component such as soy milk, soy protein, almond milk, coconut milk, or any combination thereof.

The sweetener compositions described herein may also be used in dental compositions and pharmaceutical compositions.

Further description of exemplary food, beverage, and/or other consumable products suitable for including one or more sweetener compositions described herein is disclosed in U.S. Patent Application Publication No. US2013/096420, the portions of which that disclose exemplary food, beverage, and/or other consumable compositions that can be sweetened with one or more sweetener composition described herein are incorporated herein by reference in their entirety.

When incorporated into food, beverage, and/or other consumable products, the sweetener composition may be the sole sweetening components or other sugars and/or sweeteners may also be incorporated into the product.

The sweetener compositions may also be combined with one or more additives (that may or may not also be additional sweeteners) in the food, beverage, and/or other consumable products. Examples of additives include but are not limited to carbohydrates (also as described above in additional sweeteners), polyols (also as described above in additional sweeteners), amino acids and their corresponding salts (also as described above in additional sweeteners), sugar acids and their corresponding salts, nucleotides, organic acids, inorganic acids, organic salts, including organic acid salts and organic base salts, inorganic salts, bitter compounds, caffeine, flavorants and flavoring ingredients, astringent compounds, proteins or protein hydrolysates, surfactants, emulsifiers, weighing agents, juice, dairy, cereal and other plant extracts, gums, colorants, flavonoids, alcohols, polymers, essential oils, anti-fungal agents and combinations thereof.

The sweetener compositions may also be combined with one or more bulking agents (that may or may not also qualify as additives or additional sweeteners) in the food, beverage, and/or other consumable products. Bulking agents may, for example, be used to facilitate a direct substitution of the sweetener compositions disclosed herein for sugar in applications such as baking, cooking, and tabletop uses. Examples of bulking agents include but are not limited to: a bulk sweetener such as sucrose, dextrose, invert sugar maltose, dextrin, maltodextrin, fructose, levulose, and galactose; a low glycemic carbohydrate such as fructo-oligosaccharides, galacto-oligosaccharides, mannitol, inulin, xylitol, lactitol, erythritol, and maltitol; a fiber, such as polydextrose, resistant maltodextrin, resistant starch, soluble corn fiber, and cellulose; and a hydrocolloid, such as pectin, guar gum, carboxymethylcellulose, locust bean gum, gum arabic, xanthan gum and alginate. In some embodiments, the bulking agent is selected from maltodextrin, dextro-maltodextrin blends, corn syrup solids, sucrose, fructose, glucose, invert sugar, sorbitol, xylose, ribulose, mannose, mannitol, galactitol, lactitol, isomalt, maltose, tagatose, lactose, inulin, glycerol, polydextrose, fructooligosaccharides, cellulose and cellulose derivatives or combinations thereof.

In some embodiments, the consumable products (e.g. food, beverage, and/or other consumable products) do not include nutritive sweeteners including sucrose (i.e. table sugar), glucose, fructose, corn syrup (including high fructose corn syrup), maltose, lactose, molasses, honey, agave, fruit juice, maple syrup, and others. In other embodiments, the products contain a reduced amount of the nutritive sweeteners as they are beneficially replaced (or content reduced) by the addition of the sweetener compositions (or the sweetener compound of Formula (I)) disclosed herein. In exemplary embodiments the compositions include a reduction in nutritive sweeteners of at least about 80%, 75%, 70%, 65%, 60%, 55%, 50%, 45%, 40%, 35%, 30%, 25%, 20%, 15%, 10%, or 5% in the consumable products disclosed herein.

The sweetener compositions may also be combined with one or more functional ingredients in the food, beverage, and/or other consumable products. Examples of functional ingredients include but are not limited to saponins, antioxidants, dietary fiber sources, fatty acids, vitamins, glucosamine, minerals, preservatives, hydration agents, probiotics, prebiotics, long chain primary aliphatic saturated alcohols, phytosterols and combinations thereof.

In particular embodiments, the sweetener compositions (or the sweetener compound according to Formula (I) in combination with agents described in this embodiment) are tabletop sweetener compositions. In such embodiments, the sweetener compositions (or the sweetener compound according to Formula (I) in combination with agents described in this embodiment) can further include at least one bulking agent, additive, anti-caking agent, functional ingredient and combinations thereof. The tabletop sweetener composition can be present in the form of a solid, suspension or a liquid; including co-crystallized sweetener compositions with a sugar or a polyol, agglomerated sweetener compositions, compacted sweetener compositions, dried sweetener compositions, particle sweetener compositions, spheronized sweetener compositions, spray dried compositions, granular sweetener compositions, liquid suspension compositions, and liquid sweetener compositions. Exemplary liquid tabletop sweetener can comprise water, and optionally additives, such, as for example polyols (e.g. erythritol, sorbitol, propylene glycol or glycerol), acidulants (e.g. citric acid, malic acid, tartaric acid, phosphoric acid), antimicrobial agents (e.g. benzoic acid or a salt thereof)

### Methods for Enhancing the Sugar-Like Characteristics of a Consumable Food, Beverage, or Other Product

In one embodiment, a method for imparting a more sugar-like temporal profile, flavor profile, or both to a sweetened composition or to a consumable food, beverage or other consumable product is provided. The method comprises providing a consumable food, beverage, or other product that is a sweetenable composition, and adding one or more sweetener composition described herein to such product to impart the desired degree of a sugar-like characteristic to such composition or product. The desirable characteristic can include maximal response, flavor profile, temporal profile, adaptation behavior, mouthfeel, concentration/response function behavior, tastant and flavor/sweet taste interactions, and temperature effects.

In another embodiment, one or more method for enhancing the sugar-like characteristics of a consumable food, beverage or other product described herein comprises adding one or more sweetening composition described herein to such product, wherein such sweetening composition contains a nutritive, non-nutritive and/or partially-nutritive sweetener as the additional sweetener, and where the sweetening composition provides a beneficial reduction in the sweetness onset and/or a reduce prolonged extinction time to be closer to that of sucrose. As most non-nutritive, high potency sweeteners delay the sweetness onset, the use of the sweetener compound of Formula (I) in the sweetener compositions described herein significantly reduces the sweetness onset (also referred to as appearance time) compared to a non-nutritive, high potency sweetener alone (or in a greater concentration), a desirable attribute for sweetening systems.

In another embodiment, one or more method for enhancing the sugar-like characteristics of a consumable food, beverage or other product described herein comprises adding one or more sweetening composition described herein to such product, wherein such sweetening composition contains a nutritive, non-nutritive and/or partially-nutritive sweetener as the additional sweetener, and where the sweetening composition provides a beneficial reduction of adaptation. Non-nutritive, high potency sweeteners are known to cause adaptation (a decrease in magnitude of the perceived intensity of sweetness after repeated exposure). Beneficially, one or more sweetener composition described herein do not result in any adaptation, or reduce the degree of adaptation.

In yet a further embodiment, one or more method described herein for enhancing the sugar-like characteristics of a consumable food, beverage or other product described herein comprises adding one or more sweetening composition described herein to such product, wherein said sweetener compositions suppresses, reduces or eliminates at least one undesirable taste associated with other sweetener compositions that do not contain the sweetener compound of Formula (I), wherein the at least one undesirable taste can include delayed sweetness onset, lingering sweet aftertaste, metallic taste, bitter taste, cooling sensation taste, menthol-like taste, or licorice-like taste.

In still another embodiment, one or more method described herein for enhancing the sugar-like characteristics of a consumable food, beverage or other product described herein comprises adding one or more sweetening composition described herein to such product, wherein the combination of the sweetener compound of Formula (I) with an additional sweetener beneficially possess a clean sugar-like taste with lack of undesirable off tastes, including bitterness, metal taste, astringency and/or bitter linger, while also exhibiting a sugar like temporal profile.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

The Examples described herein were tested using the sweetener compound of Formula (I), specifically Compound 5. Unless otherwise noted, all experiments were done with free base of Compound 5.

### Example 1

### Concentration-Response Testing of the Test Compound (Sweetener).

A concentration-response (CR) testing of the Test Compound was conducted using a trained descriptive analysis (DA) panel to compare the Test Compound in an aqueous matrix to sucrose. The concentration-response curve for the natural sweetener (Test Compound) was determined using a general Labeled Magnitude Scale (gLMS). Trained panelists were asked to rate the sweetness intensity of eight (8) concentrations of sweetener and sucrose, in water at room temperature (RT).

### Concentration-Response.

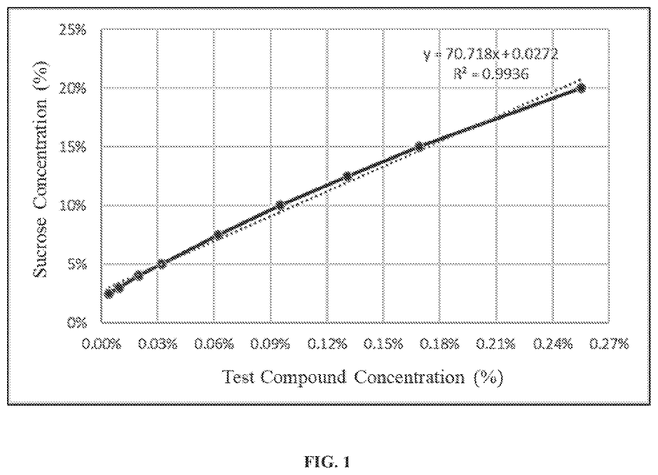

FIG. 1 shows the concentration relationship between sucrose and the Test Compound Sweetener. This relationship was established using a dose response model (Equation 2: R=Rmin+(C\hat{ }Hillslope)*(Rmax−Rmin)/(C\hat{ }HillSlope+EC50\hat{ }HillSlope) for Sucrose and the Test Compound Sweetener.

The concentration-relationship is linear for the concentration of the Test Compound Sweetener needed to achieve a target sweetness equivalence, compared to that of sucrose.

Study design and Test Samples: A trained panel of 10-12 descriptive analysis panelists participated in the experiment. All participants underwent training, during which panelists familiarized themselves with rating the sweetness intensity on the gLMS scale. The scale is depicted in FIG. 4 as a vertical line marked with anchors describing different intensities. The panelists were instructed to rate the perceived sweetness intensity for each sample by marking a line on the scale.

The order of samples was randomized and blinded using the William's Latin Square design. The order of sample presentation within each sweetener set was also randomized. All samples were evaluated using a sip and spit protocol.

The testing demonstrates the Test Compound Sweetener exhibits a linear relationship for the concentration-response curve across the tested range of concentrations; 0% to 0.2% (2000 ppm).

### Example 2

### Sensory Results of the Sweetener Blends

Sensory results for sweetener blends with Reb M were evaluated using a trained descriptive analysis (DA) panel. Five blend samples were tested in this panel; wherein each sample was designed to target a sweetness equivalence of 10 Brix Sucrose. The blends were composed of different ratios of the Test Compound Sweetener and Rebaudioside M (Reb M) in water, as shown in Table 3, to assess the sweetness quality and off-taste profile.

TABLE 3
Evaluated Sweetener Compositions

| Sample | Blend Ratios |
|---|---|
| 1 | 100% Test Compound |
| 2 | 75% Test Compound/25% Reb M |
| 3 | 50% Test Compound/50% Reb M |
| 4 | 25% Test Compound/75% Reb M |
| 5 | 100% Reb M |

Study Designs and Test Samples. A validated and trained panel of 10-12 descriptive analysis panelists participated in these experiments.

During the test sessions, panelists rated the intensity of each attribute on a 15 cm scale. Five test samples were tested for each attribute, in triplicate. The order of samples was randomized and blinded using the Williams Latin Square design. All samples were prepared in reverse osmosis (RO) water and served at room temperature and evaluated using a sip and spit protocol.

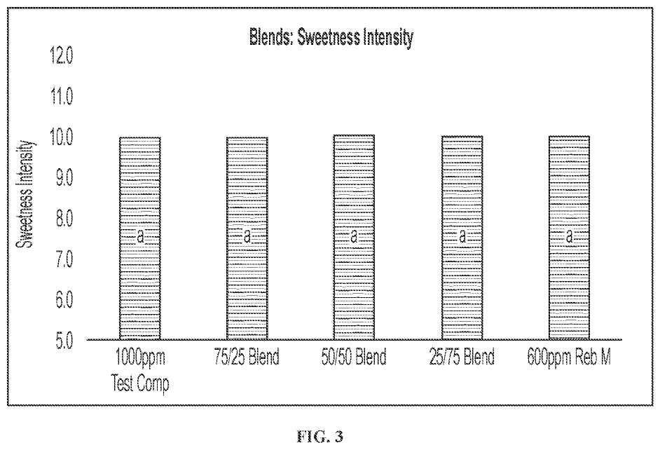

Each of the five samples were evaluated for sensory attributes corresponding to sweetness quality and off-taste profile. Attributes of sweetness intensity, sweetness appearance time, sweetness aftertaste and thickness were evaluated as part of the sweetness quality profiling. Specifically, for sweetness aftertaste two time points were evaluated; 1 minute and 3 minutes, after expectoration. The sweetness aftertaste corresponds to the sweetness linger characteristic, often common to high potency sweeteners. FIG. 3 shows that each sample achieves a sweetness intensity of “10” on a 15 cm scale (corresponding to 10 Brix sucrose) without any significant differences in sweetness intensity.

### Sweetness Profile.

Sweetness profile across the 5 blend samples was assessed where the addition of the Test Compound Sweetener to Reb M proportionally and significantly improves the desirable attributes of sweetness appearance time, sweet aftertaste and thickness. Table 4 lists the LSD Means Differences for the sweetness quality evaluation of blends with Reb M.

TABLE 4

| | 1000 ppm | | 75/25 | | 50/50 | | 25/75 | | 600 ppm | |
|---|---|---|---|---|---|---|---|---|---|---|
| Attribute | Test Comp | | Blend | | Blend | | Blend | | Reb M | |
| Sweet Intensity (ns) | 10.0 | a | 10.0 | a | 10.1 | a | 10.0 | a | 10.0 | a |
| Sweet AT* | 4.2 | d | 5.0 | c | 5.0 | c | 6.3 | b | 6.8 | a |
| Thickness | 6.7 | a | 5.1 | b | 5.1 | b | 4.0 | c | 3.5 | c |
| Sweet Aftertaste (1 min) | 3.1 | d | 4.0 | c | 4.2 | bc | 4.7 | ab | 5.1 | a |
| Sweet Aftertaste (3 min) | 0.2 | d | 0.7 | c | 0.9 | c | 1.4 | b | 1.8 | a |

*AT = Appearance Time,
ns = not significant
Different letter grouping indicates significant differences at 95% confidence level

The results in Table 3 show that increasing the proportion of the Test Compound Sweetener significantly reduces the sweetness onset, reduces the sweet linger, and increases the perceived thickness in the samples, an extremely desirable attribute for reduced sugar applications.

Off-Taste Profile. All 5 blend samples were also evaluated for attributes of bitter, astringent, metallic, licorice, and bitter aftertaste, as part of the off-taste profiling. Specifically, for bitter aftertaste two time points were evaluated; 1 minute and 3 minutes, after expectoration. When referring to ‘bitter’ tastes herein it refers to a sample that confers a bitter taste, activates or that can be detected by a bitter taste receptor and/or confers the perception of a bitter taste in a subject. Table 5 lists the LSD Means Differences for the off-taste evaluation across the 5 blend samples with Reb M.

TABLE 5

| | 1000 ppm | | 75/25 | | 50/50 | | 25/75 | | 600 ppm | |
|---|---|---|---|---|---|---|---|---|---|---|
| Attribute | Test Comp | | Blend | | Blend | | Blend | | Reb M | |
| Bitter | 1.5 | d | 2.2 | c | 2.5 | c | 3.4 | b | 4.0 | a |
| Bitter Aftertaste (1 min) | 0.2 | c | 0.9 | b | 1.0 | b | 1.4 | a | 1.6 | a |
| Bitter Aftertaste (3 min) | 0.1 | c | 0.2 | bc | 0.5 | b | 0.6 | ab | 0.9 | a |
| Astringent | 1.7 | c | 2.2 | b | 2.3 | b | 2.8 | a | 3.1 | a |
| Licorice | 1.6 | d | 2.1 | c | 2.6 | b | 2.8 | b | 3.4 | a |
| Metallic | 1.1 | d | 1.3 | c | 1.4 | bc | 1.6 | ab | 1.7 | |

Different letter grouping indicates significant differences at 95% confidence level

The results in Table 5 demonstrate that increasing the proportion of the Test Compound Sweetener in combination with Reb M significantly reduces the bitterness, bitter aftertaste, reduces the metallic off-note, licorice off-note, and the astringency off-note.

The sensory results show that in water solution, the Test Compound Sweetener by itself had minimal bitterness, astringency and bitter linger and displayed sucrose like characteristics. The inclusion of the Test Compound Sweetener in the blends resulted in significant taste improvement across multiple attributes. This taste improvement follows increasing concentrations of the Test Compound Sweetener in the blends, where samples with greater proportions of the Test Compound Sweetener show greater taste improvement. The results confirm the specific areas of significant taste improvement include sweetness appearance time, thickness, sweet aftertaste (linger), bitter, licorice, astringency, metallic and bitter aftertaste. The sweetness quality and off-taste profile of Reb M improves significantly with the addition of the Test Compound Sweetener. In addition to the taste improvement findings in these blends, these results confirm the Test Compound Sweetener is a significantly cleaner tasting sweetener than Reb M.

### Example 3

Sensory Evaluation of Test Compound Sweetener Blends

The taste profile of the Test Compound Sweetener was evaluated in combination and relative to known sweeteners; namely Rebaudioside A (Reb A), High Fructose Corn Syrup (HFCS-55), Allulose, Erythritol, Monk fruit, Rebaudioside M (Reb M) and Sucralose. The test samples are shown in Table 6 which lists contents of all samples evaluated in water. All samples were prepared by solubilizing the desired concentration of the sweetener/(s) in reverse osmosis (RO) water. Samples were tasted cold for all evaluations.

TABLE 6
Sweetener Compositions in Water

| Sample # | Content (% w/v) |
|---|---|
| 1 | 0.08% Test Compound |
| 2 | 0.055% Reb A |
| 3 | 0.05% Test Compound/0.0275% Reb A |
| 4 | 0.1% Test Compound |
| 5 | 0.05% Test Compound/5 Brix HFCS-55 |
| 6 | 10 Brix HFCS-55 |
| 7 | 3.5% Allulose |
| 8 | 0.05% Test Compound/3.5% Allulose |
| 9 | 0.017% Sucralose |
| 10 | 0.05% Test Compound/0.0085% Sucralose |
| 11 | 3.5% Erythritol |
| 12 | 0.05% Test Compound/3.5% Erythritol |
| 13 | 0.05% Monk fruit |
| 14 | 0.05% Test Compound/0.0375% Monk fruit |

These samples were evaluated by a panel (n=6) of expert tasters. Samples were tasted in a blinded fashion using sip and spit methodology. The focus of the evaluation of sweetness profile, off taste profile (described as bitter, sweet linger, sweetness onset, mouthfeel, mouth coating and metallic) and flavor profile. Qualitative descriptors for samples in water are provided in Table 7 by the panel for each sample.

TABLE 7
Qualitative Descriptors for Evaluating Sweetener Compositions in
Water

| Sample# | Sensory Description |
|---|---|
| 1 | Robust sweetness that cleans off rapidly with decent mouthfeel |
| 2 | Robust sweetness intensity with typical Reb A attributes of |
| | bitterness, sweet linger and delayed onset were observed |
| 3 | Sample was slightly sweeter than 1 and 3. Reduction in off tastes |
| | of bitterness, sweet linger and delayed onset relative to 2. |
| | Enhanced mouthfeel was observed in 3 |
| 4 | Robust sweetness that cleans off rapidly with decent mouthfeel |
| 5 | Sample showed higher sweetness intensity and mouthfeel in |
| | comparison to sample 4 and 6. |
| 6 | Robust sweetness with clean profile, no off taste with good |
| | mouthfeel |
| 7 | Very mild sweetness. Metallic and mouthcoating were significant |
| | at this sweetness intensity |
| 8 | Very robust sweetness intensity. Metallic and mouth coating |
| | were significantly reduced compared to sample 8. Mouthfeel was |
| | enhanced compared to sample 8 |
| 9 | Robust sweetness with typical sucralose notes of sharp bitter and |
| | sweet linger |
| 10 | Robust sweetness with reduction in bitter and sweetness onset. |
| | Enhanced mouthfeel relative to sample 9 |
| 11 | Very mild sweetness. Delayed onset with bitter mouth coating |
| 12 | Sample was sweeter than 11; with faster onset and reduction in |
| | bitterness relative to 10. Enhanced mouth feel was observed |
| 13 | Relatively low sweetness intensity with typical monk fruit notes |
| | of sweet linger and delayed onset |
| 14 | Higher sweetness intensity and faster onset compared to 13. |
| | Enhanced mouthfeel was observed |

Table 8 contents of all samples evaluated in tea. All samples were prepared by solubilizing the desired concentration of the sweetener/(s) in a commercial unsweetened, ready to drink (RTD) black tea. Samples were tasted cold for all evaluations

TABLE 8
Sweetener Compositions in Water

| Sample # | Content (% w/v) |
|---|---|
| 16 | 0.01% Test Compound (Na Salt) |
| 17 | 0.06% Reb M |
| 18 | 0.05% Test Compound/0.03% Reb M |

Qualitative descriptors for tea samples are provided in Table 9 by the panel for each sample.

TABLE 9
Qualitative Descriptors for Evaluating Sweetener Compositions in
Tea

| Sample# | Sensory Description |
|---|---|
| 16 | Robust sweetness that cleans off rapidly with balanced tea |
| | flavor. Enhances brewed notes in tea |
| 17 | Higher sweetness intensity compared to 16 and 18. Balanced |
| | tea flavor. Significant reduction in bitterness, sweet linger, |
| | mouth coating and sweetness onset. Enhanced mouthfeel |
| | relative to sample 18 |
| 18 | Robust sweetness intensity with significant change in flavor |
| | profile of tea. Sweetness linger, bitterness and delayed |
| | sweetness onset was observed |

The sensory evaluations of the samples in water and tea matrices demonstrate that the addition of the Test Compound Sweetener significantly improves the sweetness profile of caloric and non-caloric sweeteners by reducing the off-taste attributes. Specifically, there was reduction in bitterness, sweetness onset, sweet linger, metallic and mouth coating. It also enhances mouthfeel of the samples. These attributes observed in the blends were relative to sweeteners alone.

### Example 4

### Evaluation of Taste Profiles of Test Compound Sweetener Blends

The taste profile of the Test Compound Sweetener (Na Salt of the compound) was evaluated in combination with sweeteners—namely High Fructose Corn Syrup (HFCS-55). These samples were evaluated in pH 3.0 buffer by 3 tasters. Samples were tasted in a blinded fashion using sip and spit methodology. The focus of the evaluation was to obtain an indication of the sweetness profile of the blends when compared to sucrose. The evaluated formulations are shown in Table 10.

TABLE 10
Evaluated Sweetener Compositions

| | Test Compound Na | 50% Test Compound Na | |
|---|---|---|---|
| | Salt (1100 ppm) | Salt/50% HFCS-55 | HFCS-55 |
| Ingredient | % | % | % |
| DI Water | 99.74 | 93.31 | 86.86 |
| Citric Acid | 0.11 | 0.11 | 0.11 |
| Sodium Citrate | 0.02 | 0.02 | 0.02 |
| Potassium Sorbate | 0.02 | 0.02 | 0.02 |
| Test Compound Na Salt | 0.11 | 0.06 | — |
| HFCS-55 (77 brix) | — | 6.49 | 12.99 |
| Total | 100 | 100 | 100 |

Materials and Methods. Potassium sorbate was dissolved into water. The remaining ingredients were added and mixed until fully dissolved. The pH of each sample was recorded and the samples tasted using sip and spit method. The samples were then compared to each other.

Results. The Test Compound at 1100 ppm is fairly clean in taste and provides good upfront sweetness. It was lower in mouthfeel. The combination of 550 ppm Test Compound+6.49% HFCS-55 had similar sweetness and slightly more mouthfeel than the Test Compound alone. HFCS-55 at 12.987% has similar sweetness and is very clean.

The results show that the Test Compound was pretty clean in sweetness with good onset and decay, but lacked mouthfeel compared to HFCS-55. The testing confirms the Test Compound offers a sugar like taste profile along with HFCS and moves the product closer to sucrose.

### Example 5

### Evaluation of Taste Profiles of Test Compound Sweetener Blends

The taste profile of the Test Compound Sweetener (Na Salt of the compound) was evaluated in combination with sweeteners—namely Rebaudioside A (Reb A). Enliten® 300000 (Reb A 95) was used in combination with the Test Compound. These samples were evaluated in pH 3.0 buffer by 3 tasters. Samples were tasted in a blinded fashion using sip and spit methodology. The focus of the evaluation was to obtain an indication of the sweetness profile of the blends when compared to sucrose. The evaluated formulations are shown in Table 11.

TABLE 11
Evaluated Sweetener Compositions

| | | Test | 50% Test | |
|---|---|---|---|---|
| | | Compound Na | Compound Na | Reb A |
| | HFCS-55 | Salt (880 ppm) | Salt/50% Reb A | (550 ppm) |
| Ingredient | % | % | % | % |
| DI Water | 89.46 | 99.76 | 99.78 | 99.80 |
| Citric Acid | 0.11 | 0.11 | 0.11 | 0.11 |
| Sodium Citrate | 0.02 | 0.02 | 0.02 | 0.02 |
| Potassium Sorbate | 0.02 | 0.02 | 0.02 | 0.02 |
| HFCS-55 (77 brix) | 10.39 | — | — | — |
| Test Compound Na | — | 0.088 | 0.04 | — |
| Salt | | | | |
| Enliten ® 300000 | — | — | 0.03 | 0.06 |
| (Reb A 95) | | | | |
| Total | 100.000 | 100.000 | 100.000 | 100.000 |

Materials and Methods. Potassium sorbate was dissolved into water. The remaining ingredients were added and mixed until fully dissolved. The pH of each sample was recorded and the samples tasted using sip and spit method. The samples were then compared to each other.

Results. The Test Compound Na Salt at 880 ppm is fairly clean in taste and provides similar sweetness. It has good onset and decay, but has a slight salty note at the end. It lacks mouthfeel compared to HFCS-55, but it provides some mouthfeel compared to Reb A. The combination of 440 ppm Test Compound Na Salt+275 ppm Reb A may be slightly sweeter than 8SE. Addition of Test Compound reduced the off notes commonly seen with Reb A. Reb A at 550 ppm is much less sweet than 8SE. It has a plastic/floral/metallic taste to it with a bitter and astringent linger. This is the worst tasting sample of the group with regards to off notes.

The results confirm that Test Compound Na Salt is pretty clean in sweetness with good onset and decay. It provides slightly more mouthfeel than Reb A and can help in reducing some of the off notes commonly seen in Reb A while not reducing sweetness. The Test Compound helped reduce the adaptation commonly with Reb A.

### Example 6

### Evaluation of Taste Profiles of Test Compound Sweetener Blends

The taste profile of the Test Compound Sweetener (Na Salt of the compound) was evaluated in combination with sweeteners—namely Allulose. Astraea® Liquid Allulose was used in combination with the Test Compound. These samples were evaluated in pH 3.0 buffer at varying sweetness equivalence (SE) (also may be referred to as sucrose sweetness equivalence (SSE)) targets by 3 tasters. Samples were tasted in a blinded fashion using sip and spit methodology. The focus of the evaluation was to obtain an indication of the sweetness profile of the blends when compared to sucrose. The evaluated formulations are shown in Table 12.

TABLE 12
Evaluated Sweetener Compositions

| | 5 SE | | 7.5 SE | | 12.5 SE | |
|---|---|---|---|---|---|---|
| | | Test Compound | | Test Compound | | Test Compound |
| | HFCS-55 | Na Salt + Allulose | HFCS-55 | Na Salt + Allulose | HFCS-55 | Na salt + Allulose |
| Ingredient | % | % | % | % | % | % |
| DI Water | 93.36 | 95.11 | 90.11 | 95.09 | 83.62 | 95.03 |
| Citric Acid | 0.11 | 0.11 | 0.11 | 0.11 | 0.11 | 0.11 |
| Sodium Citrate | 0.02 | 0.02 | 0.02 | 0.02 | 0.02 | 0.02 |
| Potassium Sorbate | 0.02 | 0.02 | 0.02 | 0.02 | 0.02 | 0.02 |
| HFCS-55 (77 brix) | 6.49 | — | 9.74 | — | 16.23 | — |
| Test Compound Na Salt | — | 0.03 | — | 0.06 | — | 0.11 |
| Astraea ® Liquid | — | 4.71 | — | 4.71 | — | 4.71 |
| Allulose (74 Brix) | | | | | | |
| Total | 100. | 100 | 100 | 100 | 100 | 100 |

Materials and Methods. Potassium sorbate was dissolved into water. The remaining ingredients were added and mixed until fully dissolved. The pH of each sample was recorded and the samples tasted using sip and spit method. The samples were then compared to each other.

Results. At 5 SE, the combination of the Test Compound Na Salt and Astraea® Liquid Allulose is similar in sweetness to the HFCS target at 5SSE. It was slightly less sour in taste, but there were virtually no differences seen in bitterness, mouthfeel and aftertaste. At 7.5 SE, the combination of the Test Compound Na Salt and Astraea® Liquid Allulose is close in sweetness to the HFCS target. Mouthfeel decreased slightly, but all other attributes were comparable. At 12.5 SE, the combination of the Test Compound Na Salt and Astraea® Liquid Allulose is close in sweetness to the HFCS target. Bitterness increased very slightly and mouthfeel decreased very slightly, but aftertaste remained comparable.

The testing confirms that the Test Compound Na Salt with Astraea® Liquid Allulose has the ability to replace HFCS at a wide range of sweetness equivalence targets. They provide a clean, sweet taste when paired together with no major off notes. Mouthfeel differences may be noticeable as the sweetness equivalence increases. The combination of the Test Compound and allulose offers a “sugar like” taste profile and moves the product closer to sucrose

### Example 7

### Evaluation of Taste Profiles of Test Compound Sweetener Blends

The taste profile of the Test Compound Sweetener (Na Salt of the compound) was evaluated in combination with sweeteners—namely sucralose. These samples were evaluated in pH 3.0 buffer at 10 SE by 3 tasters. Samples were tasted in a blinded fashion using sip and spit methodology. The focus of the evaluation was to obtain an indication of the sweetness profile of the blends when compared to sucrose. The evaluated formulations are shown in Table 13.

TABLE 13
Evaluated Sweetener Compositions

| | | | 50% Test | |
|---|---|---|---|---|
| | | Test Compound | Compound Na | |
| | | Na Salt | Salt/50% | Sucralose |
| | HFCS-55 | (1100 ppm) | Sucralose | (550 ppm) |
| Ingredient | % | % | % | % |
| DI Water | 86.86 | 99.74 | 99.79 | 99.83 |
| Citric Acid | 0.11 | 0.11 | 0.11 | 0.11 |
| Sodium Citrate | 0.02 | 0.02 | 0.02 | 0.02 |
| Potassium Sorbate | 0.02 | 0.02 | 0.02 | 0.02 |
| HFCS-55 (77 | 12.99 | — | — | — |
| brix) | | | | |
| Test Compound | — | 0.11 | 0.06 | — |
| Na Salt | | | | |
| Sucralose | — | — | 0.01 | 0.02 |
| Total | 100 | 100 | 100 | 100 |

Materials and Methods. Potassium sorbate was dissolved into water. The remaining ingredients were added and mixed until fully dissolved. The samples were tasted using sip and spit method. The samples were then compared to each other and the HFCS-55 sweetness equivalence target (10 SE).

Results. The Test Compound Na Salt at 1100 ppm is less sweet than HFCS (LOSE), but is similar in sweetness to Sucralose at 170 ppm. The Test Compound Na Salt is slightly lower in mouthfeel with a slightly sour and salty linger. The combination of 550 ppm Test Compound Na Salt+85 ppm Sucralose is less sweet than HFCS (LOSE). The combination of the two sweeteners and Test Compound Na Salt at 1100 ppm was closer in taste quality to HFCS. Less aftertaste/linger than Sucralose at 170 ppm was observed. Sucralose by itself at 170 ppm is less sweet than HFCS (LOSE) with a delayed sweetness. Significant aftertaste/linger was detected.

The testing confirms the Test Compound Na Salt with Sucralose may help reduce the aftertaste/linger of using Sucralose alone at similar sweetness equivalence. The Test Compound helped reduce the adaptation commonly with sucralose.

It is to be understood that while the invention has been described in conjunction with the detailed description thereof, the foregoing description is intended to illustrate, and not limit the scope of the invention, which is defined by the scope of the appended claims. Other embodiments, advantages, and modifications are within the scope of the following claims. The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

1. A sweetener composition comprising:
(a) at least one compound of Formula (I): 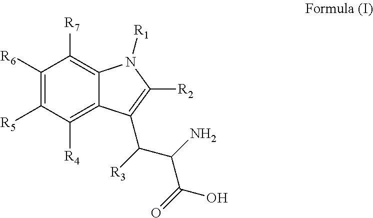
or a comestibly or biologically acceptable salt or stereoisomeric form thereof, wherein:
R1 is hydrogen or C1-C6 alkyl;
R2-R5 and R7 are each independently hydrogen, halogen, C1-C6 alkyl, C1-C6 alkoxy, hydroxyl or trifluoromethyl; and
R6 is I, hydrogen, C4-C6 alkyl, C4-C6 alkoxy or hydroxyl,
wherein the salt is optionally selected from a sodium salt, a potassium salt, ammonium salt, or a calcium salt,
wherein the sweetener composition contains an effective amount of the compound of Formula (I) to provide a sucrose sweetness equivalence of at least 1%; and
(b) at least one additional sweetener comprising a zero calorie sweetener, a fully caloric sweetener and/or a low calorie sweetener,
wherein the sweetener composition provides more sugar-like characteristics than a sweetener composition without the compound of Formula (I).

2. The sweetener composition of claim 1, wherein R1, R2, R3, R5, R6, and R7 are independently at each occurrence hydrogen, and wherein R4 is methyl or wherein R1, R2, R3, R4, R6, and R7 are independently at each occurrence hydrogen, and wherein R5 is methyl; or wherein R1, R3, R4, R5, R6, and R7 are independently at each occurrence hydrogen, and wherein R2 is methyl.

3-4. (canceled)

5. The sweetener composition of claim 1, wherein the at least one compound of Formula (I) is selected from 2-amino-3-(4-methyl-1H-indol-3-yl)propanoic acid, 2-amino-3-(5-methyl-1H-indol-3-yl)propanoic acid, (R)-2-amino-3-(5-methyl-1H-indol-3-yl)propanoic acid, (S)-2-amino-3-(5-methyl-1H-indol-3-yl)propanoic acid, and 2-amino-3-(2-methyl-1H-indol-3-yl)propanoic acid.

6. The sweetener composition of claim 5, wherein the compound of Formula (I) comprises a racemic mixture of the at least one compound, or wherein the compound of Formula (I) is the D-isomer.

7. The sweetener composition of claim 1, wherein the compound of Formula (I) is one or more of the following formulae:

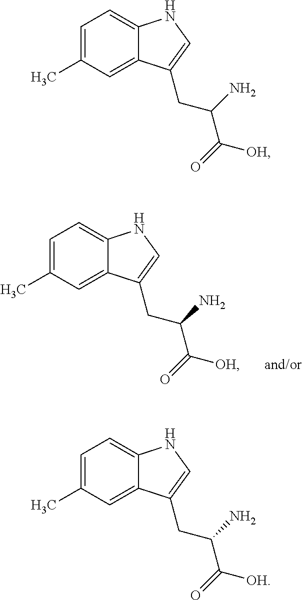 [SMILES: Cc1ccc2[nH]cc(CC(N)C(=O)O)c2c1 ; Cc1ccc2[nH]cc(C[C@@H](N)C(=O)O)c2c1 ; Cc1ccc2[nH]cc(C[C@H](N)C(=O)O)c2c1]

8. (canceled)

9. The sweetener composition of claim 1, wherein the sweetener compound of Formula (I) comprises at most 0.1%, 0.5%, 1%, 2.5%, 5%, 7.5%, 10%, 25%, 50%, 75%, 90%, or 95% by weight of the composition.

10. The sweetener composition of claim 1, wherein the sugar-like characteristic is selected from maximal response, flavor profile, temporal profile, adaptation behavior, mouthfeel, concentration/response function behavior, tastant and flavor/sweet taste interactions, and temperature effects.

11. The sweetener composition of claim 1, where the additional sweetener is a zero calorie, high potency sweetener comprising one or more steviol glycosides or a fully caloric sweetener comprising a carbohydrate sweetener or a low calorie sweetener comprising allulose or a polyol or a combination thereof.

12. The sweetener composition of claim 1, wherein the additional sweetener is
(i) a zero calorie, high potency sweetener comprising one or more of rebaudioside A (Reb A), rebaudioside B (Reb B), rebaudioside C (Reb C), rebaudioside D (Reb D), rebaudioside D4 (Reb D4), rebaudioside E (Reb E), rebaudioside F (Reb F), rebaudioside G (Reb G), rebaudioside H (Reb H), rebaudioside I (Reb I), rebaudioside J (Reb J), rebaudioside K (Reb K), rebaudioside L (Reb L), rebaudioside M2 (Reb M2), rebaudioside M (Reb M), rebaudioside N (Reb N), rebaudioside O (Reb O), rebaudioside S (Reb S), rebaudioside T (Reb T), rebaudioside U (Reb U), rebaudioside V (Reb V), rebaudioside W (Reb W), rebaudioside Z1 (Reb Z1), rebaudioside Z2 (Reb Z2), amino acid, tryptophan, steviolmonoside, steviolbioside, dulcoside A, dulcoside B, rubusoside, stevia, stevioside, mogroside, mogroside IV, mogroside V, mogroside VI, iso-mogroside V, grosmomoside, neomogroside, siamenoside, Luo Han Guo sweetener, siamenoside, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobtain, baiyunoside, osladin, polypodoside A, pterocaryoside A, pterocaryoside B, mukurozioside, phlomisoside I, periandrin I, abrusoside A, and cyclocarioside I, or
(ii) is selected from Reb A, Reb B, Reb D, Reb M, Reb J, stevioside, mogroside, erythritol, allulose, high fructose corn syrup (HFCS), sucrose, cane sugar, aspartame, acesulfame-potassium, sucralose, and any combination of two or more of the foregoing.

13-15. (canceled)

16. The sweetener composition of claim 1, wherein the sweetener compositions can also suppress, reduce or eliminate at least one undesirable taste associated with other sweeteners not containing the sweetener compound of Formula (I), wherein the at least one undesirable taste can include delayed sweetness onset, lingering sweet aftertaste, metallic taste, bitter taste, cooling sensation taste, menthol-like taste, or licorice-like taste.

17. The sweetener composition of claim 1, wherein the sweetener composition further provides a beneficial reduction of adaptation, wherein adaptation is a decrease in magnitude of perceived intensity of sweetness after repeated exposure to a sweetener.

18. The sweetener composition of claim 1, wherein the sweetener composition has a clean sweet taste with lack of undesirable off-tastes, wherein the off-taste comprises one or more of bitterness, metal taste, astringency and/or bitter linger.

19. A consumable food, beverage, or other product comprising:
a concentration from about 5 ppm to about 4000 ppm of the sweetener composition of claim 1;
at least one additive, bulking agent and/or functional ingredient; and
a food, beverage, or other consumable composition.

20. The consumable product of claim 19, wherein the sweetener compound has the following structure:

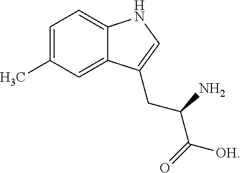 [SMILES: Cc1ccc2[nH]cc(C[C@@H](N)C(=O)O)c2c1]

21. The consumable product of claim 19, wherein the consumable product is a beverage product, beverage concentrate, a food product, a dietary sweetener, dental product, and/or pharmaceutical product.

22. (canceled)

23. The consumable product of claim 19, wherein the consumable product is a food or beverage having an equivalent sweetness to sucrose solutions having concentrations between 10,000 and 400,000 ppm (1-40SSE).

24. A consumable product comprising:
a concentration from about 5 ppm to about 4000 ppm of at least one compound of Formula (I): 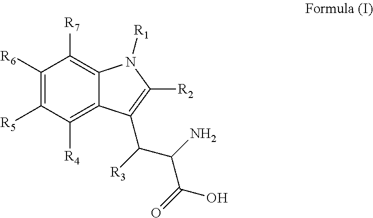
or a comestibly or biologically acceptable salt or stereoisomeric form thereof, wherein:
R1 is hydrogen or C1-C6 alkyl;
R2-R5 and R7 are each independently hydrogen, halogen, C1-C6 alkyl, C1-C6 alkoxy, hydroxyl or trifluoromethyl; and
R6 is I, hydrogen, C4-C6 alkyl, C4-C6 alkoxy or hydroxyl;
a product selected from a beverage product and a consumable
product; and
an additional compound selected from Reb A, Reb B, Reb D, Reb M, mogroside V, erythritol, allulose, high fructose corn syrup (HFCS), aspartame, sucralose acesulfame-potassium K, Saccharin, Cyclamate, amino acids, and combination thereof;
wherein the consumable product has an equivalent sweetness to sucrose solutions having concentrations between 10,000 and 400,000 ppm (1-40SSE);

25. The consumable product of claim 24, wherein the compound of Formula (I) provides a sucrose sweetness equivalence of at least 1%, at least 5%, at least 10%, at least 15%, or at least 20%.

26. The consumable product of claim 24, wherein the product is selected from pharmaceutical compositions, edible gel mixes and compositions, dental compositions, confections, condiments, chewing gum, cereal compositions, frozen foods, desserts, soy based products, protein products, snacks, dressings, sauces, gravy, baked goods, dairy products, nondairy products, tabletop sweeteners, food, non-alcoholic beverages, alcoholic beverages and beverage products, fruits preparations and concentrates, dressings, frozen food, canned food, desserts, bars, snacks, sauces, and spreads.

27. The consumable product of claim 24, wherein the product is a beverage product or a mid-calorie, low-calorie or zero-calorie beverage.

28-32. (canceled)
